(19) United States
(12) Patent Application Publication
(10) Pub. No.: US 2026/0265819 A1
(43) Pub. Date: Sep. 10, 2026

(54) UNTARGETED MULTIPLEXED IN SITU RNA PROFILING AND THE USES AND MEANS THEREFOR

(71) Applicants: The Broad Institute, Inc., Cambridge, MA (US); Massachusetts Institute of Technology, Cambridge, MA (US)

(72) Inventors: Xiao WANG, Cambridge, MA (US); Xin SUI, Cambridge, MA (US)

(73) Assignees: The Broad Institute, Inc., Cambridge, MA (US); Massachusetts Institute of Technology, Cambridge, MA (US)

(21) Appl. No.: 19/566,292

(22) Filed: Mar. 13, 2026

Related U.S. Application Data
(63) Continuation of application No. PCT/US2024/046583, filed on Sep. 13, 2024.
(60) Provisional application No. 63/583,037, filed on Sep. 15, 2023.

(51) Int. Cl. C12Q 1/6869; C12N 15/10; C12Q 1/34; C12Q 1/6806; C12Q 1/6841; C12Q 1/6855

(52) U.S. Cl. CPC C12Q 1/6869; C12N 15/1096; C12Q 1/34; C12Q 1/6806; C12Q 1/6841; C12Q 1/6855; C12Q 2600/16; G01N 2333/922

(57) ABSTRACT

The present disclosure provides methods (referred to herein as “SWITCH-seq”), compositions, kits, and systems for profiling RNA expression in a cell (including, e.g., cells within an intact tissue) in both untargeted and targeted manners. Also provided by the present disclosure are methods for diagnosing a disease or disorder in a subject based on a profile of RNA expression in a cell tissue, or other biological sample. Methods of screening for or testing a candidate agent capable of modulating RNA expression are also provided by the present disclosure. The present disclosure also provides methods for treating a disease or disorder in a subject in need thereof. Oligonucleotides useful for performing the methods described herein are also provided by the present disclosure. Additionally, the present disclosure provides kits comprising any combination of the oligonucleotides described herein.

### RELATED APPLICATIONS

This application is a continuation of International Application No. PCT/US2024/046583, filed on Sep. 13, 2024, and claims priority under 35 U.S.C. § 119 (e) to U.S. Provisional Application No. 63/583,037, filed on Sep. 15, 2023. The entire contents of the aforementioned patent applications are incorporated herein by reference.

### REFERENCE TO AN ELECTRONIC SEQUENCE LISTING

The instant application contains a Sequence Listing which has been filed electronically in XML format and is hereby incorporated by reference in its entirety. Said XML file, created on Mar. 11, 2026, is named 114203-3023_SL.xml and is 43,404 Bytes in size.

### BACKGROUND OF THE INVENTION

RNA molecules serve as the bridge between DNA and proteins, playing pivotal roles in the regulation of gene expression, cellular function, and organismal development. From messenger RNAs (mRNAs) encoding genetic blueprints for proteins, to non-coding RNAs, such as ribosomal RNAs (rRNAs), transfer RNAs (tRNAs), and microRNAs that modulate diverse cellular pathways, the vast array and intricacy of RNA types highlight their essential and varied roles in cellular biology. Probing the specific subcellular locales of these RNAs is pivotal to deciphering the nuanced relationships between RNA dynamics, spatial distribution, and their functional roles.

Targeted in situ RNA mapping methods, such as targeted in situ sequencing¹⁻³ or multiplexed fluorescence in situ hybridization (FISH),⁴⁻⁶ enable precise subcellular assessment of gene expression. However, these methods require predetermined gene sets for measurement and possess inherent limitations in their ability to discern single-base variations within RNA sequences.

Contrastingly, untargeted in situ RNA sequencing methodologies allow for the comprehensive analysis of a broad array of RNAs within specific cellular and tissue environments, underscoring the potential to identify spatially distinct sequence variants. However, recent optical-based methods, exemplified by FISSEQ⁷, face challenges with limited detection efficiency^{2,8} Several alternative approaches involve interrogating location information of RNA, such as by transferring RNA to barcoded microparticles.⁹⁻¹² However, these strategies necessitate the use of high-end or tailor-made equipment and result in a loss of subcellular resolution and precision. Therefore, additional technologies and systems for untargeted in situ RNA sequencing are needed.

### SUMMARY OF THE INVENTION

The present disclosure describes SWITCH-seq, a template-switching-based multiplexed in situ RNA sequencing technique that is adept at profiling a wide range of RNA species (e.g., mRNA, rRNA) and their sequence variants with enhanced efficiency and specificity. Notably, SWITCH-seq offers versatility, accommodating both untargeted sequencing (without confinement to a predetermined gene list) and targeted in situ sequencing. SWITCH-seq possesses the capacity to identify single-base variants within cells and intact tissue specimens (e.g., at subcellular resolution).

Thus, in one aspect, the present disclosure provides methods, uses, compositions, kits, and systems for profiling RNA expression in a cell or multiple cells, including cells in a fixed tissue sample or other biological sample (see, for example, FIG. 1A). In the methods and systems disclosed herein, a cell may be contacted with a population of primer probes, a template switching oligonucleotide (TSO), and a reverse transcriptase. Following reverse transcription and circularization, the resulting cDNAs can be amplified (e.g., by rolling circle amplification) to produce concatenated amplicons. The concatenated amplicons may then be embedded in a polymeric matrix and sequenced to determine the identity of the transcripts and their location within the polymeric matrix (e.g., through SEDAL sequencing (Sequencing with Error-reduction by Dynamic Annealing and Ligation) as described further herein). Using the locations of the modified transcripts, RNAs expression may be profiled in either a targeted manner (i.e., with primer probes comprising a known sequence that is complementary to one or more RNAs of interest) or an untargeted manner (i.e., with primer probes comprising random sequences that hybridize to RNAs of unknown sequences), and spatiotemporal information may be obtained to improve the understanding of how RNA expression affects cellular function in health and disease. The methods and systems may be useful for comparing RNA expression in, for example, a cell (or multiple cells) from diseased and healthy tissue samples; or for comparing RNA expression in, for example, a cell treated with an agent (e.g., a therapeutic agent or potential therapeutic agent, such as a small molecule, a protein, a peptide, a nucleic acid, a lipid, or a carbohydrate) and an untreated cell, or a diseased cell and a healthy cell. In some embodiments, the methods provided herein comprise additional steps so they can be used to profile RNAs that are bound to a ribosome.

In some embodiments, the present disclosure provides methods for profiling RNA expression in a cell comprising the steps of:
a) contacting the cell with a population of primer probes, wherein each primer probe in the population comprises a first oligonucleotide portion that is complementary to a portion of an RNA in the cell, and a second oligonucleotide portion that is not complementary to the RNA;
b) contacting the cell with a reverse transcriptase, wherein the reverse transcriptase uses the first oligonucleotide portion of each of the primer probes to reverse transcribe the RNA to which each primer probe is hybridized, thereby producing a corresponding cDNA for each RNA;
c) contacting the cell with a template switching oligonucleotide (TSO), wherein a portion of the TSO is complementary to the 3′ end of each cDNA (e.g., to additional untemplated nucleotides, such as deoxycytidines, added to the cDNA by the reverse transcriptase as described herein), and wherein the reverse transcriptase uses a portion of the TSO that is not complementary to the cDNA as a template to add the reverse complement sequence of the portion of the TSO to the 3′ end of the cDNA;
d) contacting the cell with an RNase, wherein the RNase digests all or substantially all of the RNA in the cell;
e) ligating the 5′ end and the 3′ end of each cDNA together to produce circular cDNA molecules;
f) performing rolling circle amplification to amplify the circular cDNA molecules, thereby producing a population of concatenated amplicons;
g) embedding the concatenated amplicons in a polymeric matrix; and
h) sequencing the concatenated amplicons, or a portion thereof, embedded in the polymeric matrix to determine the identity and location of RNAs in the cell.

These methods can thus be used to determine the expression patterns and locations of RNAs within a cell or population of cells (e.g., cells in an intact tissue), or within organelles of a cell. In some embodiments, the RNAs comprise known sequences of interest. In some embodiments, the RNAs are of unknown sequences, and the method is used to profile their expression in an untargeted manner. In some embodiments, more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 RNAs of interest are profiled simultaneously using the methods described herein.

The methods, compositions, kits, and systems described herein may be useful for studying RNA expression in tissues (e.g., developing tissues, normal tissues, diseased tissues, treated tissues), for diagnosing and treating various diseases, for research purposes, for drug discovery, and for any other purposes recognized by one of skill in the art. Thus, in one aspect, the present disclosure provides methods for diagnosing a disease or disorder in a subject. For example, the methods for profiling RNA expression described herein may be performed on a cell, or on multiple cells, taken from a subject (e.g., a subject who is thought to have or is at risk of having a disease or disorder, or a subject who is healthy or thought to be healthy). The expression of various RNAs in the cell(s) can then be compared to the expression of the same or other RNAs in one or more non-diseased cells or one or more cells from a non-diseased tissue sample (e.g., a cell from a healthy individual, or multiple cells from a population of healthy individuals). Any difference in the RNA expression profile of the cell (including of a single RNA or of multiple RNAs, e.g., a specific disease signature) relative to one or more non-diseased cells may indicate that the subject has the disease or disorder. RNA expression in one or more non-diseased cells (e.g., normal cells) may be profiled alongside expression in a diseased cell as a control experiment. RNA expression in one or more non-diseased cells (e.g., normal cells) may have also been profiled previously, and the profile of a diseased cell may be compared to reference data for a non-diseased cell (e.g., a normal cell).

In another aspect, the present disclosure provides methods of screening for an agent (e.g., a therapeutic agent, or any kind of stimulus such as a mechanical force, light, heat, electricity, etc.) capable of modulating RNA expression. For example, the methods for profiling RNA expression described herein may be performed in a cell in the presence of one or more candidate agents. The expression of various RNAs in the cell (e.g., a normal cell, or a diseased cell) can then be compared to the expression of the RNAs in a cell that was not exposed to the one or more candidate agents. Any difference in the RNA expression profile relative to the cell that was not exposed to the candidate agent(s) may indicate that expression of particular RNAs is modulated by the candidate agent(s). In some embodiments, a particular signature (e.g., of altered expression of multiple RNAs) that is known to be associated with the treatment of a disease may be used to identify agents capable of modulating RNA expression in a desired manner and thus treating a disease. The methods and systems described herein may also be used to identify drugs that have certain side effects, for example, by looking for particular RNA expression signatures associated with a side effect when one or more cells is treated with a candidate agent or known drug (or combinations of multiple candidate agents and/or known drugs, e.g., as provided in a screening library of compounds). The methods and systems described herein may also be used to identify research reagents or chemical probes that may be useful for studying RNA expression, RNA location, RNA processing, RNA mutations (e.g., single base substitutions), etc.

In another aspect, the present disclosure provides methods for treating a disease or disorder in a subject. For example, the methods and systems for profiling RNA expression described herein may be performed in a cell from a sample taken from a subject (e.g., a subject who is thought to have or is at risk of having a disease or disorder). The RNA expression profile can then be compared to the RNA expression profile of a cell from a non-diseased tissue sample. A treatment for the disease or disorder (e.g., a pharmaceutical agent, surgery, radiation therapy, surgery, physical therapy, lifestyle changes, etc.) may then be administered to the subject if any difference in the RNA expression profile relative to a non-diseased cell is observed. RNA expression in one or more non-diseased cells may be profiled alongside RNA expression in a diseased cell as a control experiment. RNA expression in one or more non-diseased cells (e.g., normal cells) may have also been profiled previously, and RNA expression in a diseased cell may then be compared to reference data for a non-diseased cell.

In another aspect, the present disclosure provides sets of oligonucleotide probes comprising: i) a primer probe comprising a first oligonucleotide portion that is complementary to a portion of an RNA in a cell, and a second oligonucleotide portion that is not complementary to the RNA; and ii) a template switching oligonucleotide (TSO). In some embodiments, the sets of oligonucleotide probes further comprise iii) a circularization probe, wherein a portion of the circularization probe is complementary to the second oligonucleotide portion of the primer probe, and wherein another portion of the circularization probe is complementary to the reverse complement of a portion of the TSO.

In another aspect, the present disclosure provides pluralities of oligonucleotides comprising multiple sets of oligonucleotides as provided herein, wherein each set of oligonucleotides comprises a primer probe that is complementary to a different RNA in a cell. In certain embodiments, the plurality comprises more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of oligonucleotides.

In another aspect, the present disclosure provides kits (e.g., a kit comprising any of the sets of oligonucleotides or pluralities of oligonucleotides disclosed herein). In some embodiments, the kit comprises multiple sets of oligonucleotides as described herein, each of which can be used to identify a specific RNA. In certain embodiments, the kit comprises more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of oligonucleotides. The kits described herein may also include any other reagents or components useful in performing the methods described herein, including, but not limited to, enzymes (such as a ligase, a polymerase (e.g., a reverse transcriptase and/or a DNA polymerase), and/or an RNase), nucleotides comprising a nucleophile (e.g., amine-modified nucleotides), buffers, reagents (including dyes, stains, and more), and monomers for making a polymeric matrix (e.g., a polyacrylamide matrix).

In another aspect, the present disclosure provides compositions comprising one or more cDNAs, concatenated amplicons, and/or polymeric matrix-embedded concatenated amplicons produced by any of the methods described herein. In some embodiments, the composition includes parts or remnants of cells and/or tissues.

In another aspect, the present disclosure provides systems for profiling RNA expression in a cell. In some embodiments, such a system comprises:
a) a cell;
b) one or more primer probes, wherein each primer probe comprises a first oligonucleotide portion that is complementary to a portion of an RNA in the cell and a second oligonucleotide portion that is not complementary to the RNA;
c) a reverse transcriptase; and
d) a template switching oligonucleotide (TSO).

Any of the oligonucleotides (i.e., the sets and pluralities of oligonucleotides) described herein may be used in the systems contemplated by the present disclosure. In some embodiments, the system further comprises an RNase. In some embodiments, the system further comprises a DNA ligase. In some embodiments, the system further comprises a DNA polymerase. In some embodiments, the system further comprises nucleotides comprising a nucleophile (e.g., amine-modified nucleotides). In some embodiments, the system further comprises reagents and monomers for preparing a polymeric matrix. In some embodiments, the system further comprises a microscope. In some embodiments, the system further comprises a computer. In some embodiments, the system further comprises a liquid handling system.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the Detailed Description of Specific Embodiments presented herein.

FIGS. 1A-1D show design and validation of SWITCH-seq. FIG. 1A provides a schematic summary of SWITCH-seq: following cell fixation, a reverse transcriptase (RT) primer containing random octamers and a 5′ flanking sequence anneal to the RNA. In tandem with a template-switching oligonucleotide (TSO), cDNA synthesis occurs, integrating a known sequence (the reverse complement of the TSO) at the 3′ end. RNase digestion subsequently removes any residual RNA, after which a circularization probe facilitates splint ligation. Then, rolling circle amplification (RCA) is conducted to construct the in situ cDNA amplicons. These amplicons are then copolymerized with acrylamide, leading to formation of a DNA-gel hybrid (wavy lines). This integration is enabled by the functionalized acrylic group, incorporated in a previous modification step. Finally, the RNA sequence is deciphered through sequencing with error-reduction by dynamic annealing and ligation (SEDAL). FIG. 1B shows the rationale for the design of SWITCH-seq. The generation of an amplicon necessitates the presence of an RT primer, TSO, and circularization probes. FIG. 1C provides schematics and representative raw fluorescent images of HeLa cells illustrating SWITCH-seq and respective negative control experiments, showing that the methodology selectively amplifies RNA sequences that are reverse transcribed with the TSO's integration at the 3′ end, followed by circularization. All four images show amplicons in HeLa cells (DAPI). FIG. 1D shows quantitative analysis of cDNA amplicons. Error bars represent standard deviation. For each condition, n=4 images. Student's t-test, P<0.01.

FIGS. 2A-2D show that SWITCH-seq discerns single-base variations in rRNA. FIG. 2A provides a schematic summary of SWITCH-seq, tailored for rRNA variant mapping. Following cell fixation, a specifically designed RT primer targets designated rRNA regions and, together with a 5′ flanking sequence, anneals to the rRNA. In tandem with a TSO, cDNA synthesis occurs, integrating a known sequence (the reverse complement of the TSO) to the 3′ end. These cDNAs encapsulate the rRNA variants (dots). RNase digestion subsequently removes any residual RNA, after which a circularization probe facilitates splint ligation. Then RCA is conducted to construct the in situ cDNA amplicons. These amplicons are then copolymerized with acrylamide, leading to formation of a DNA-gel hybrid (wavy lines). This integration is enabled by the functionalized acrylic group, incorporated in a previous modification step. Finally, the rRNA variant is decoded through SEDAL sequencing. FIG. 2B shows two rounds of representative fluorescent in situ sequencing images of HeLa cells (DAPI) for the es39l-probed region. A non-variable base C (underlined) was identified at position 4912. At position 4913, two alternative sequences were revealed: the known reference sequence C and the alternative variant U. FIG. 2C provides representative fluorescent images of HeLa cells (DAPI) showcasing three highly abundant rRNA variants. The positions of the variants are indicated at the bottom of the images, while the reference and alternative alleles are indicated at the top, along with their respective rRNA frequencies.

FIG. 2D, left, provides representative raw fluorescent images of HeLa cells (DAPI) showcasing both SWITCH-seq and FISSEQ experiments mapping the rRNA variant at the es39l position 4913. Both images show two sequence alternatives: the recognized reference sequence C and the alternate variant U. FIG. 2D, right, shows quantitative analysis of amplicons. Error bars represent standard deviation. For each condition, n=4 images.

FIGS. 3A-3C show optimization of template switching oligos. FIG. 3A, left, provides representative raw fluorescent images of HeLa cells (DAPI) showcasing SWITCH-seq using DNA, RNA, and a DNA/RNA hybrid as the TSO. Amplicons are shown as spots in and surrounding the cells. FIG. 3A, right, shows quantitative analysis of amplicons. Error bars represent standard deviation. For each condition, n=4 images. FIG. 3B provides a schematic overview of SWITCH-seq utilizing hairpin-structured TSO. Following cell fixation, an RT primer containing random octamers and a 5′ flanking hairpin sequence anneal to the RNA. In tandem with a hairpin TSO, cDNA synthesis occurs, integrating a known sequence (the reverse complement of the TSO) to the 3′ end. RNase digestion subsequently removes any residual RNA. Gap-filling is then performed to bridge the space between the two hairpin configurations. Ligation follows to circularize the cDNA, and then RCA is used to construct the in situ cDNA amplicons. These amplicons are then copolymerized with acrylamide, leading to formation of a DNA-gel hybrid (wavy lines). This integration is enabled by the functionalized acrylic group, incorporated in a previous modification step. Finally, the RNA sequence is deciphered through SEDAL sequencing. FIG. 3C, left, provides representative raw fluorescent images of HeLa cells (DAPI) showcasing SWITCH-seq using linear and hairpin TSO. Amplicons are shown as spots in and surrounding the cells. FIG. 3C, right, shows quantitative analysis of template switching efficiency as assessed by qPCR. The C_{q} value (quantification cycle) in qPCR refers to the cycle number at which the fluorescence of a PCR product crosses a predefined threshold, indicating the presence and relative abundance of the target sequence.

FIGS. 4A-4C show optimization of other aspects of SWITCH-seq. FIG. 4A, left, shows quantitative analysis of template switching efficiency using an RNA template with different chemical caps, as assessed by qPCR. FIG. 4A, right, shows the chemical structures of different chemical caps. FIG. 4B shows quantitative analysis of template switching efficiency using different RNase inhibitors, as assessed by qPCR. FIG. 4C provides qPCR serial dilution curves used to evaluate the efficiency of primers used in template switching assays.

FIG. 5 shows analysis of SWITCH-seq specificity. Left: Authentic SWITCH-seq signals. Middle: Non-specific SWITCH-seq signals. Right: FISSEQ non-specific signals due to self-circularization, manifesting as abundant amplicons in regions absent of cells.

FIGS. 6A-6F show applications of SWITCH-seq in spatial translatome profiling. FIG. 6A shows a modified SWITCH-seq strategy using an rRNA-splint to achieve untargeted in situ ribosome profiling. FIG. 6B shows that a biotinylated cDNA fixer strand fixes cDNA in place after streptavidin incubation and bis-succinimide (PEG)₉ (BS(PEG)₉) crosslinking to prevent cDNA migration. FIGS. 6C-6D show recruitment of the RT enzyme to ribosome-bound mRNA to produce cDNA for SWITCH-seq library preparation. FIG. 6E provides representative images showing amplicons of the original SWITCH-seq (global splint ligation) and rRNA-splint ligation in HeLa cells. FIG. 6F shows that rRNA-splint ligation enriches more coding RNA (Actin beta (ACTB) gene) compared to non-coding RNA (Metastasis Associated Lung Adenocarcinoma Transcript 1 (MALAT1) gene).

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “administer,” “administering,” and “administration” refer to implanting, absorbing, ingesting, injecting, inhaling, or otherwise introducing a treatment or therapeutic agent, or a composition of treatments or therapeutic agents, in or on a subject.

The term “amplicon” as used herein refers to a nucleic acid (e.g., RNA or DNA) that is the product of an amplification reaction (i.e., the production of one or more copies of a genetic fragment or target sequence) or replication reaction. Amplicons can be formed artificially using, for example, PCR or other polymerization reactions. The term “concatenated amplicons” refers to multiple amplicons that are joined together to form a single nucleic acid molecule. Concatenated amplicons can be formed, for example, by rolling circle amplification (RCA), in which a circular oligonucleotide is amplified to produce multiple linear copies of the oligonucleotide as a single nucleic acid molecule comprising multiple amplicons that are concatenated.

The term “cDNA” refers to DNA that is derived from (e.g., by reverse transcription) and complementary to an RNA template (e.g., an mRNA template or an rRNA template). In some embodiments, a cDNA comprises additional nucleotides at the 3′ end that are not derived from an RNA template. In certain embodiments, a cDNA comprises additional deoxycytidines at the 3′ end that are not derived from an RNA template.

A “cell,” as used herein, may be present in a population of cells (e.g., in a tissue, a sample, a biopsy, an organ, or an organoid). In some embodiments, a population of cells is composed of a plurality of different cell types. Cells for use in the methods and systems of the present disclosure can be present within an organism, a single cell type derived from an organism, or a mixture of cell types. Included are naturally occurring cells and cell populations, genetically engineered cell lines, cells derived from transgenic animals, cells from a subject, etc. Virtually any cell type and size can be accommodated in the methods and systems described herein. In some embodiments, the cells are mammalian cells (e.g., complex cell populations such as naturally occurring tissues). In some embodiments, the cells are from a human. In certain embodiments, the cells are collected from a subject (e.g., a human) through a medical procedure, such as a biopsy. Alternatively, the cells may be a cultured population (e.g., a culture derived from a complex population or a culture derived from a single cell type where the cells have differentiated into multiple lineages). The cells may also be provided in situ in a tissue sample.

Cell types contemplated for use in the methods and systems of the present disclosure include, but are not limited to, stem and progenitor cells (e.g., embryonic stem cells, hematopoietic stem cells, mesenchymal stem cells, neural crest cells, etc.), endothelial cells, muscle cells, myocardial cells, smooth and skeletal muscle cells, mesenchymal cells, epithelial cells, hematopoietic cells, lymphocytes such as T-cells (e.g., Th1 T cells, Th2 T cells, ThO T cells, cytotoxic T cells) and B cells (e.g., pre-B cells), monocytes, dendritic cells, neutrophils, macrophages, natural killer cells, mast cells, adipocytes, immune cells, neurons, hepatocytes, and cells involved with particular organs (e.g., thymus, endocrine glands, pancreas, brain, neurons, glia, astrocytes, dendrocytes, and genetically modified cells thereof). The cells may also be transformed or neoplastic cells of different types (e.g., carcinomas of different cell origins, lymphomas of different cell types, etc.) or cancerous cells of any kind (e.g., from any of the cancers disclosed herein). Cells of different origins (e.g., ectodermal, mesodermal, and endodermal) are also contemplated for use in the methods and systems of the present disclosure. In some embodiments, the cells are microglia, astrocytes, oligodendrocytes, excitatory neurons, or inhibitory neurons. In some embodiments, the cells are cardiac cells. In certain embodiments, the cells are HeLa cells. In some embodiments, cells of multiple cell types are present within the same sample. In certain embodiments, the cells are from a diseased tissue sample or diseased subject. In certain embodiments, the cells are from a healthy tissue sample or healthy subject. In some embodiments, a cell is from a cell line. In certain embodiments, a cell is from any of the following cell lines: 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells.

The term “complementary” is used herein to refer to two oligonucleotide sequences (e.g., DNA or RNA) comprising bases that hydrogen bond to one another. The degree of complementarity between two oligonucleotide sequences can vary, from complete complementarity to no complementarity (e.g., 100% complementarity, 99% complementarity, 98% complementarity, 97% complementarity, 96% complementarity, 95% complementarity, 90% complementarity, 85% complementarity, 80% complementarity, or less than 80% complementarity). For example, two oligonucleotide sequences may be only partially complementary to one another (e.g., in the probes described herein, wherein only a portion of the probe is complementary to a nucleotide sequence). In some embodiments, a sequence is complementary to only a portion of another sequence. In some embodiments, a sequence is complementary to another sequence under certain conditions (e.g., certain salt concentrations, pHs, etc.).

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA and mean any chain of two or more nucleotides. The polynucleotides can be chimeric mixtures or derivatives or modified versions thereof, and single-stranded or double-stranded. The oligonucleotide can be modified at the base moiety, sugar moiety, or phosphate backbone, for example, to improve stability of the molecule, its hybridization parameters, etc.

The term “profiling” (in reference to profiling RNA expression in the methods provided herein) refers to determining the expression pattern of multiple (potentially thousands of) genes at once to create a global picture of cellular function. An RNA expression profile includes information about which genes are and are not expressed by a particular cell at a particular time point. An RNA expression profile also includes information about the levels at which genes are expressed in the cell. The RNA expression profile of a cell can be used, for example, to determine the cell type of the cell, the stage of cell division the cell is at, whether the cell is from a diseased or healthy tissue, or how the cell responds to treatment with a particular agent.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. A protein may also be a therapeutic protein administered as a treatment for a disease or disorder (e.g., one that is associated with a change in the RNA expression profile of a cell taken from a subject). In certain embodiments, the protein is an antibody, or an antibody variant (including antibody fragments).

The term “reverse transcriptase” refers to a class of polymerases that are capable of using a primer to synthesize a DNA sequence from an RNA template. For example, a reverse transcriptase can transcribe an mRNA transcript into cDNA. In particular, the present disclosure contemplates the use of reverse transcriptases that are capable of appending additional non-templated nucleotides (e.g., one or more deoxycytidines) at the 3′ end of a cDNA prior to terminating reverse transcription. In some embodiments, the reverse transcriptase is an MMLV reverse transcriptase, or a variant thereof. In some embodiments, the reverse transcriptase is a wild type MMLV reverse transcriptase. In some embodiments, the reverse transcriptase is an MMLV reverse transcriptase comprising one or more amino acid substitutions relative to a wild type MMLV reverse transcriptase.

A “transcript” or “RNA transcript” is the product resulting from RNA polymerase-catalyzed transcription of a DNA sequence. When the RNA transcript is a complementary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell.

The term “sample” or “biological sample” refers to any sample including tissue samples (such as tissue sections, surgical biopsies, and needle biopsies of a tissue); cell samples; or cell fractions, fragments, or organelles (such as obtained by lysing cells and separating the components thereof by centrifugation or otherwise). Other examples of biological samples include, but are not limited to, blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, a biological sample is a surgical biopsy taken from a subject, for example, a biopsy of any of the tissues described herein. In certain embodiments, a biological sample is a tumor biopsy. In some embodiments, the sample is brain tissue. In some embodiments, the tissue is cardiac tissue. In some embodiments, the sample is epithelial tissue, connective tissue, muscular tissue, or nervous tissue. In some embodiments, the sample is tissue from the central nervous system (e.g., brain). In some embodiments, the cells used in the methods described herein come from such a sample or biological sample.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey) or mouse). The term “patient” refers to a subject in need of treatment of a disease. In some embodiments, the subject is human. In some embodiments, the patient is human. The human may be a male or female at any stage of development. A subject or patient “in need” of treatment of a disease or disorder includes, without limitation, those who exhibit any risk factors or symptoms of a disease or disorder. In some embodiments, a subject is a non-human experimental animal (e.g., a mouse, rat, dog, or non-human primate).

The term “template switching” refers to a process in which a polymerase (such as a reverse transcriptase) utilizes a first nucleic acid template to synthesize a nucleic acid molecule and subsequently uses a second nucleic acid template to append additional nucleotides on the end of the same nucleic acid molecule prior to termination of polymerization. To accomplish this, a “template switching oligonucleotide (TSO)” is used in the methods provided herein. The TSO comprises a portion that is complementary to the 3′ end of a cDNA (e.g., complementary to a sequence of untemplated deoxycytidines added to the cDNA during reverse transcription). A reverse transcriptase then uses a further portion of the TSO that is not complementary to the cDNA as a template to add the reverse complement sequence of this portion of the TSO to the 3′ end of the cDNA.

The term “therapeutic agent,” as used herein, refers to any agent that can be used to treat a disease or disorder, or reduce or alleviate the symptoms of a disease or disorder. In some embodiments, the therapeutic agent is a small molecule, a protein, a peptide, a nucleic acid, a lipid, or a carbohydrate. In some embodiments, the therapeutic agent is a known drug and/or an FDA-approved drug. In certain embodiments, the protein is an antibody. In certain embodiments, the protein is an antibody variant. In certain embodiments, the protein is a receptor, or a fragment or variant thereof. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), or an antisense oligonucleotide (ASO).

A “therapeutically effective amount” of a treatment or therapeutic agent is an amount sufficient to provide a therapeutic benefit in the treatment of a condition or to delay or minimize one or more symptoms associated with the condition. A therapeutically effective amount of a treatment or therapeutic agent means an amount of the therapy, alone or in combination with other therapies, that provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

As used herein, a “tissue” is a group of cells and their extracellular matrix from the same origin. Together, the cells carry out a specific function. The association of multiple tissue types together forms an organ. The cells may be of different cell types. In some embodiments, a tissue is an epithelial tissue. Epithelial tissues are formed by cells that cover an organ surface (e.g., the surface of the skin, airways, soft organs, reproductive tract, and inner lining of the digestive tract). Epithelial tissues perform protective functions and are also involved in secretion, excretion, and absorption. Examples of epithelial tissues include, but are not limited to, simple squamous epithelium, stratified squamous epithelium, simple cuboidal epithelium, transitional epithelium, pseudostratified epithelium, columnar epithelium, and glandular epithelium. In some embodiments, a tissue is a connective tissue. Connective tissues are fibrous tissues made up of cells separated by non-living material (e.g., an extracellular matrix). Connective tissues provide shape to organs and hold organs in place. Connective tissues include fibrous connective tissue, skeletal connective tissue, and fluid connective tissue. Examples of connective tissues include, but are not limited to, blood, bone, tendon, ligament, adipose, and areolar tissues. In some embodiments, a tissue is a muscular tissue. Muscular tissue is an active contractile tissue formed from muscle cells. Muscle tissue functions to produce force and cause motion. Muscle tissue includes smooth muscle (e.g., as found in the inner linings of organs), skeletal muscle (e.g., as typically attached to bones), and cardiac muscle (e.g., as found in the heart, where it contracts to pump blood throughout an organism). In some embodiments, a tissue is a nervous tissue. Nervous tissue includes cells comprising the central nervous system and peripheral nervous system. Nervous tissue forms the brain, spinal cord, cranial nerves, and spinal nerves (e.g., motor neurons). In certain embodiments, a tissue is brain tissue.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed (e.g., prophylactically or upon suspicion or risk of disease). In other embodiments, treatment may be administered in the absence of signs or symptoms of the disease. For example, treatment may be administered to a susceptible subject prior to the onset of symptoms (e.g., in light of a history of symptoms in the subject, or family members of the subject). Treatment may also be continued after symptoms have resolved, for example, to delay or prevent recurrence. In some embodiments, treatment may be administered after using the methods disclosed herein and observing a change in the RNA expression profile in a cell or tissue in comparison to a healthy cell or tissue.

### DETAILED DESCRIPTION OF CERTAIN EMBODIMENTS

The aspects described herein are not limited to specific embodiments, systems, compositions, methods, kits, uses, or configurations, and as such can, of course, vary. The terminology used herein is for the purpose of describing particular aspects only and, unless specifically defined herein, is not intended to be limiting.

The present disclosure provides methods, uses, compositions, kits, and systems for profiling RNA expression in a cell, or in multiple cells (e.g., cells present within an intact tissue, or isolated cells). In particular, the methods, uses, compositions, kits, and systems provided herein may, in some embodiments, be used to profile RNA expression in an untargeted manner. The present disclosure also provides methods for diagnosing a disease or disorder in a subject based on a profile of RNA expression in a cell, including cells within an intact tissue. The present disclosure also provides methods for treating a disease or disorder in a subject in need thereof. Methods of screening for or testing a candidate agent capable of modulating RNA expression are also provided by the present disclosure. Oligonucleotides useful for performing the methods described herein are also provided by the present disclosure, as well as kits comprising any of the oligonucleotides described herein.

### Methods for Profiling RNA Expression

In one aspect, the present disclosure provides methods for profiling RNA expression in a cell (or in multiple cells, e.g., in an intact tissue). Such methods are useful for profiling RNA expression in both untargeted and targeted manners. In the methods disclosed herein, a cell is contacted with a population of primer probes, a template switching oligonucleotide (TSO), and a reverse transcriptase. Following the reverse transcription and template switching process, the resulting cDNAs are circularized and amplified to produce concatenated amplicons. The concatenated amplicons may then be embedded in a polymeric matrix and sequenced to determine the identity of the transcripts and their location within the polymeric matrix (e.g., through SEDAL sequencing as described further herein).

In some embodiments, the present disclosure provides methods for profiling RNA expression in a cell comprising the steps of:
a) contacting the cell with a population of primer probes, wherein each primer probe in the population comprises a first oligonucleotide portion that is complementary to a portion of an RNA in the cell and a second oligonucleotide portion that is not complementary to the RNA;
b) contacting the cell with a reverse transcriptase, wherein the reverse transcriptase uses the first oligonucleotide portion of each of the primer probes to reverse transcribe the RNA to which each primer probe is hybridized, thereby producing a corresponding cDNA for each RNA;
c) contacting the cell with a template switching oligonucleotide (TSO), wherein a portion of the TSO is complementary to the 3′ end of each cDNA, and wherein the reverse transcriptase uses a portion of the TSO that is not complementary to the cDNA as a template to add the reverse complement sequence of the portion of the TSO to the 3′ end of the cDNA;
d) contacting the cell with an RNase, wherein the RNase digests all or substantially all of the RNA in the cell;
e) ligating the 5′ end and the 3′ end of each cDNA together to produce circular cDNA molecules;
f) performing rolling circle amplification to amplify the circular cDNA molecules, thereby producing a population of concatenated amplicons;
g) embedding the concatenated amplicons in a polymeric matrix; and
h) sequencing the concatenated amplicons, or a portion thereof, embedded in the polymeric matrix to determine the identity and location of RNAs in the cell.

The methods described herein utilize a population of primer probes for priming reverse transcription of RNAs in a cell. Each primer probe in the population comprises a first oligonucleotide portion that is complementary to a portion of an RNA in the cell and a second oligonucleotide portion that is not complementary to the RNA. In some embodiments, each primer probe comprises the structure 5′-[second oligonucleotide portion not complementary to RNA]-[first oligonucleotide portion complementary to RNA]-3′, wherein]-[represents an optional linker (e.g., a nucleotide linker). In some embodiments,]-[represents a direct linkage between the two portions of the primer probe (i.e., a phosphodiester bond). In some embodiments, the first oligonucleotide portion of the primer probes comprises DNA. In some embodiments, the second oligonucleotide portion of the primer probes comprises DNA. In certain embodiments, both the first and the second oligonucleotide portions of the primer probes comprise DNA. In some embodiments, the first oligonucleotide portion of the primer probes comprises RNA. In some embodiments, the second oligonucleotide portion of the primer probes comprises RNA. In certain embodiments, both the first and the second oligonucleotide portions of the primer probes comprise RNA. In some embodiments, the primer probes comprise modified nucleotides or nucleotide analogs.

In some embodiments, the methods provided herein may be used to profile RNA expression in an untargeted manner. In such embodiments, the first oligonucleotide portion of each of the primer probes comprises an unknown sequence (i.e., a sequence of random nucleotides). The unknown sequence of each primer probe is thus capable of hybridizing to a random RNA sequence in the cell, thereby allowing for untargeted reverse transcription. In some embodiments, the population of primer probes comprises primer probes with 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 6000 or more, 7000 or more, or 8000 or more random nucleotide sequences. In some embodiments, the first oligonucleotide portion of each of the primer probes comprises a random sequence of 5-12, 6-11, 7-10, or 8-9 nucleotides in length. In some embodiments, the first oligonucleotide portion of each of the primer probes comprises a random sequence of 5, 6, 7, 8, 9, 10, 11, or 12 nucleotides in length. In certain embodiments, the first oligonucleotide portion of each of the primer probes comprises a random sequence of 8 nucleotides in length.

In some embodiments, the methods provided herein may be used to profile RNA expression in a targeted manner. For example, the methods may be utilized to profile expression of RNAs with one or more mutations of interest (e.g., rRNAs comprising single base variants). In such embodiments, the first oligonucleotide portion of each of the primer probes comprises a known sequence. In some embodiments, the known sequence is complementary to a non-variable region of an RNA. In certain embodiments, the known sequence is complementary to a non-variable region of an rRNA. In some embodiments, the known sequence comprises a poly dT sequence. In some embodiments, the first oligonucleotide portion of each of the primer probes comprises a known sequence of 8-40, 9-39, 10-38, 11-37, 12-36, 13-35, 14-34, 15-33, 16-32, 17-31, 18-30, 19-29, 20-28, 21-27, 22-26, or 23-25 nucleotides in length. In some embodiments, the first oligonucleotide portion of each of the primer probes comprises a known sequence of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the first oligonucleotide portion of each of the primer probes comprises a known sequence with a melting temperature of 40-75° C., 41-74° C., 42-73° C., 43-72° C., 44-71° C., 45-70° C., 46-69° C., 47-68° C., 48-67° C., 49-66° C., or 50-65° C. In certain embodiments, the first oligonucleotide portion of each of the primer probes comprises a known sequence with a melting temperature of 50-65° C.

In some embodiments, the second oligonucleotide portion of each primer probe comprises a known sequence. For example, the second oligonucleotide portion of each primer probe may comprise a known sequence at the 5′end in order to facilitate circularization of cDNAs produced in the methods described herein (i.e., a portion of the circularization probes described herein may be complementary to such a known sequence). In some embodiments, the second oligonucleotide portion of each primer probe is 20-40, 21-39, 22-38, 23-37, 24-36, 25-35, or 26-34 nucleotides in length. In some embodiments, the second oligonucleotide portion of each primer probe is 20, 21, 22, 23, 24, 25, 26, 27, 28, 29, 30, 31, 32, 33, 34, 35, 36, 37, 38, 39, or 40 nucleotides in length. In certain embodiments, the second oligonucleotide portion of each primer probe is 27 nucleotides in length. In some embodiments, the second oligonucleotide portion of each of the primer probes comprises a sequence at least 90%, at least 95%, at least 96%, at least 97%, at least 98%, or at least 99% identical to the sequence TATACACTAAAGATAGGATCCACT (SEQ ID NO: 1). In certain embodiments, the second oligonucleotide portion of each of the primer probes comprises the sequence TATACACTAAAGATAGGATCCACT (SEQ ID NO: 1).

In some embodiments, the second oligonucleotide portion of each of the primer probes comprises a secondary structural motif at the 5′end. In some embodiments, the secondary structural motif is a hairpin sequence. In certain embodiments, the hairpin sequence comprises a sequence at least 90%, at least 95%, at least 96%, at least 97%, at least 98%, or at least 99% identical to the sequence CACCGAGACTGCGAGTCACACATACACTAAAGCTTGGGACAGACTACCTCTCTC GCAGTCTCGGT (SEQ ID NO: 2). In certain embodiments, the hairpin sequence comprises the sequence CACCGAGACTGCGAGTCACACATACACTAAAGCTTGGGACAGACTACCTCTCTC GCAGTCTCGGT (SEQ ID NO: 2).

The methods of the present disclosure also contemplate the use of a reverse transcriptase enzyme to produce corresponding cDNAs for any RNA sequence to which a primer probe utilized in the methods hybridizes. In particular, the present disclosure contemplates the use of any reverse transcriptase that is capable of appending additional non-templated nucleotides at the 3′ end of a cDNA prior to terminating reverse transcription. In some embodiments, the reverse transcriptase appends additional non-templated nucleotides at the 3′ end of each cDNA prior adding the reverse complement of the TSO to the 3′ end of the cDNA. In some embodiments, the additional non-templated nucleotides comprise one or more deoxycytidines. In certain embodiments, the additional non-templated nucleotides comprise two, three, four, or five deoxycytidines.

In some embodiments, the reverse transcriptase is an MMLV reverse transcriptase, or a variant thereof. In some embodiments, the reverse transcriptase is a wild type MMLV reverse transcriptase. In some embodiments, the reverse transcriptase is an MMLV reverse transcriptase comprising at least one, at least two, at least three, at least four, at least five, at least six, at least seven, at least eight, at least nine, or at least ten amino acid substitutions relative to a wild type MMLV reverse transcriptase. The present disclosure also contemplates the use of commercially available reverse transcriptase variants, and in particular, those that have been optimized for use in template switching procedures. In some embodiments, the reverse transcriptase is a commercially available MMLV reverse transcriptase variant. In some embodiments, the reverse transcriptase comprises the sequence: AFPLERPDWDYTTQAGRNHLVHYRQLLLAGLQNAGRSPTNLAKVKGITQGPNESPS AFLERLKEAYRRYTPYDPEDPGQETNVSMSFIWQSAPDIGRKLGRLEDLKSKTLGDL VREAEKIFNKRETPEEREERIRRETEEKEERRRTVDEQKEKERDRRRHREMSKLLATV VIGQEQDRQEGERKRPQLDKDQCAYCKEKGHWAKDCPKKPRGPRGPRPQTSLLTLG DXGGQGQDPPPEPRITLKVGGQPVTFLVDTGAQHSVLTQNPGPLSDKSAWVQGATG GKRYRWTTDRKVHLATGKVTHSFLHVPDCPYPLLGRDLLTKLKAQIHFEGSGAQVV GPMGQPLQVLTLNIEDEYRLHETSKEPDVSLGFTWLSDFPQAWAESGGMGLAVRQA PLIIPLKATSTPVSIKQYPMSQEARLGIKPHIQRLLDQGILVPCQSPWNTPLLPVKKPGT NDYRPVQDLREVNKRVEDIHPTVPNPYNLLSGLPPSHQWYTVLDLKDAFFCLRLHPT SQPLFAFEWRDPEMGISGQLTWTRLPQGFKNSPTLFDEALHRDLADFR (SEQ ID NO: 3) or a sequence at least 80%, at least 85%, at least 90%, at least 95%, at least 96%, at least 97%, at least 98%, or at least 99% to this sequence.

In some embodiments, the methods provided herein further comprise washing the cell with an RNase inhibitor prior to contacting the cell with the reverse transcriptase. In some embodiments, the RNase inhibitor is a small molecule. In some embodiments, the RNase inhibitor is an enzyme. In some embodiments, the RNase inhibitor is RNaseIN™ Plus, RNaseOUT™, or SUPERnaseIn™. In certain embodiments, the RNase inhibitor is RNaseOUT™. In some embodiments, a nucleotide analog is provided to the cell along with the reverse transcriptase and is thereby incorporated into the cDNAs during reverse transcription. In some embodiments, the method further comprises crosslinking the nucleotide analog-modified cDNAs to one another prior to RNase digestion. In certain embodiments, the nucleotide analog is a nucleotide comprising a nucleophile. In certain embodiments, the nucleotide analog is an amine-modified nucleotide. In certain embodiments, the nucleotide analog is aminoallyl-dUTP.

The methods described herein also contemplate the use of a template switching oligonucleotide (TSO). Template switching is a process in which a polymerase (such as a reverse transcriptase) utilizes a first template to produce a nucleic acid molecule and subsequently switches to a second template that is used to append additional templated nucleotides on the end of the same nucleic acid molecule. To accomplish this, a portion of the TSO used in the methods provided herein is complementary to the 3′ end of each cDNA, and the reverse transcriptase then uses a further portion of the TSO that is not complementary to the cDNA as a template to add the reverse complement sequence of this portion of the TSO to the 3′ end of the cDNA. In some embodiments, the portion of the TSO that is complementary to the 3′ end of each cDNA is complementary to the additional non-templated nucleotides added by the reverse transcriptase at the 3′ end of the cDNA (e.g., a sequence of untemplated deoxycytidines). In some embodiments, the portion of the TSO that is complementary to the 3′ end of each cDNA comprises one or more guanosines and/or deoxyguanosines. In some embodiments, the portion of the TSO that is complementary to the 3′ end of each cDNA comprises two, three, four, or five guanosines and/or deoxyguanosines. In certain embodiments, the portion of the TSO that is complementary to the 3′ end of each cDNA comprises three guanosines and/or deoxyguanosines. In some embodiments, the TSO comprises DNA. In some embodiments, the TSO comprises RNA. In certain embodiments, the TSO comprises a hybrid of DNA and RNA. In some embodiments, the TSO comprises modified nucleotides or nucleotide analogs. In some embodiments, the TSO comprises a secondary structural motif. In certain embodiments, the secondary structural motif is a hairpin sequence. In some embodiments, the TSO is 15-50 nucleotides in length. In some embodiments, the TSO is 21-42 nucleotides in length. In certain embodiments, the TSO is 21, 22, 23, 24, 25, 26, 27, 28, 29, 30, 31, 32, 33, 34, 35, 36, 37, 38, 39, 40, 41, or 42 nucleotides in length.

In some embodiments, the TSO comprises a sequence at least 90%, at least 95%, at least 96%, at least 97%, at least 98%, or at least 99% identical to any one of the sequences TACCAACGCAGAGTACATrGrGG (SEQ ID NO: 4), rUrArCrCrArArCrGrCrArGrArGrUrArCrArUrGrGG (SEQ ID NO: 5), rUrArCrCrArACGCrArGrArGrUrACATrGrGG (SEQ ID NO: 6), and AACCGAGACTGCGAGAAATACCCNNNNNCATCCCTGCCAAGCTTCTAACTCGCA GTCTCGGTTTCGTAGACTAAGATrGrGG (SEQ ID NO: 7). In certain embodiments, the TSO comprises the sequence TACCAACGCAGAGTACATIGrGG (SEQ ID NO: 4), rUrArCrCrArArCrGrCrArGrArGrUrArCrArUrGrGG (SEQ ID NO: 5), rUrArCrCrArACGCrArGrArGrUrACATrGrGG (SEQ ID NO: 6), or AACCGAGACTGCGAGAAATACCCNNNNNCATCCCTGCCAAGCTTCTAACTCGCA GTCTCGGTTTCGTAGACTAAGATrGrGG (SEQ ID NO: 7). The designation “r” prior to a nucleotide in these sequences or any sequences provided herein designates that the following nucleotide is a ribonucleotide, while nucleotides not marked with “r” are deoxyribonucleotides.

The methods provided herein also comprise a step of ligating the 5′ end and the 3′ end of each cDNA together to produce circular DNA molecules for use in rolling circle amplification. In some embodiments, the step of ligating the 5′ end and the 3′ end of each cDNA together comprises contacting the cell with a circularization probe. The circularization probes used herein comprise a portion that is complementary to the second oligonucleotide portion of the primer probe (which comprises a known sequence), and an additional portion that is complementary to the reverse complement of the portion of the TSO that was added to the 3′ end of each cDNA. In this way, the circularization probe brings the 5′ end and the 3′ end of each cDNA molecule in close proximity to one another to facilitate their ligation into a circular molecule. In some embodiments, ligating the 5′ end and the 3′ end of each cDNA together further comprises contacting the cell with a DNA ligase.

In some embodiments, the circularization probe is 30-70, 31-69, 32-68, 33-67, 34-66, 35-65, 36-64, 37-63, 38-62, 39-61, 40-60, 41-59, 42-58, 43-57, 44-56, or 45-55 nucleotides in length. In some embodiments, the circularization probe is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length. In certain embodiments, the circularization probe is 48 nucleotides in length. In some embodiments, the circularization probe comprises a sequence at least 90%, at least 95%, at least 96%, at least 97%, at least 98%, or at least 99% identical to the sequence AGTGGATCCTATCTTTAGTGTATATACCAACGCAGAGTACAT (SEQ ID NO: 8). In certain embodiments, the circularization probe comprises the sequence AGTGGATCCTATCTTTAGTGTATATACCAACGCAGAGTACAT (SEQ ID NO: 8).

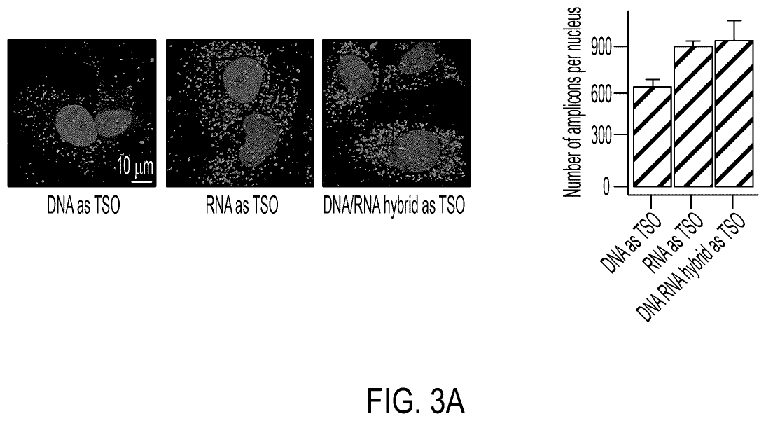

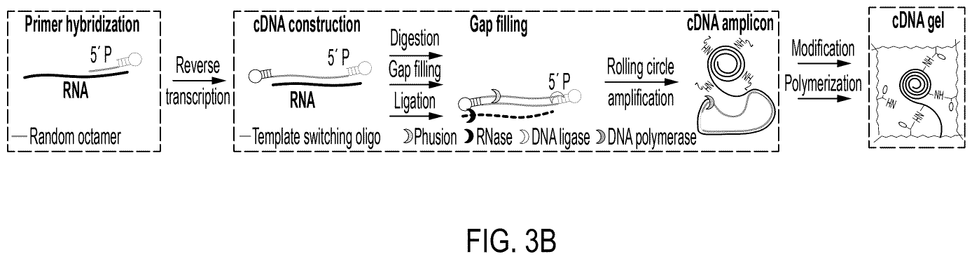

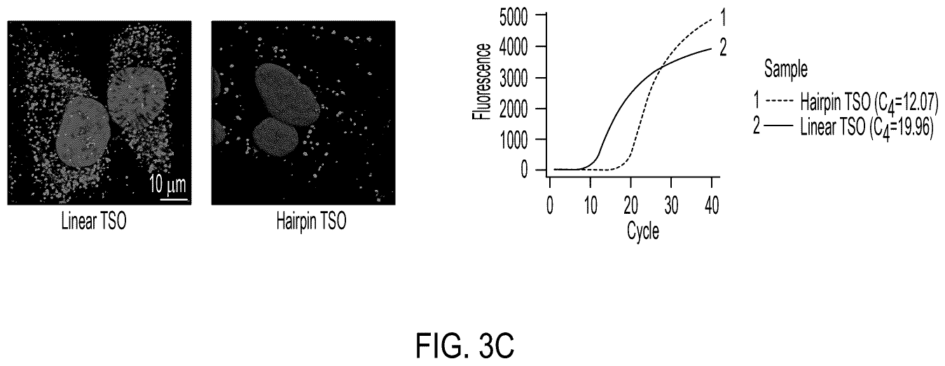

In some embodiments in which the primer probes and the TSO comprise a secondary structural motif (e.g., a hairpin sequence) as described herein, ligating the 5′ end and the 3′ end of each cDNA together may comprise gap-filling the space between the secondary structural motif (e.g., hairpin sequence) of the second oligonucleotide portion of the primer probe and the secondary structural motif (e.g., hairpin sequence) of the TSO that has been added to the 3′ end of each cDNA by contacting the cell with a DNA polymerase. The DNA polymerase thereby fills in the gap between each hairpin sequence (see, for example, FIG. 3B). In certain embodiments, the DNA polymerase utilized in the gap-filling procedure is Phusion® High-Fidelity DNA Polymerase. In certain embodiments, ligating the 5′ end and the 3′ end of each cDNA together further comprises contacting the cell with a DNA ligase.

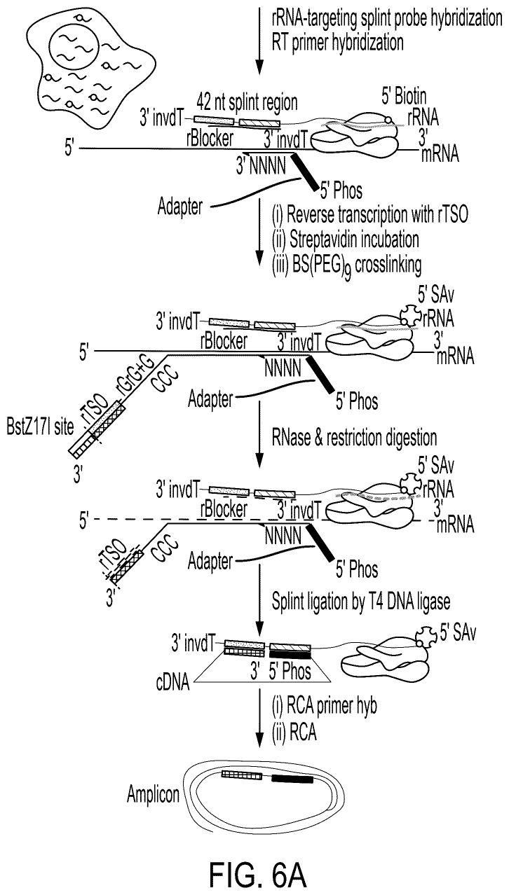

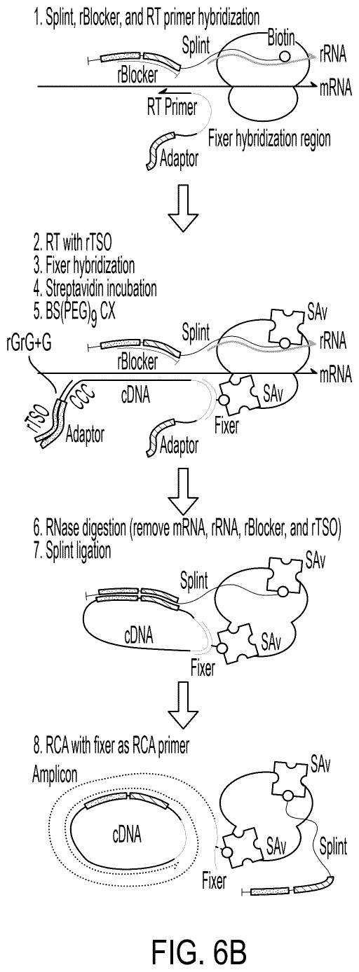

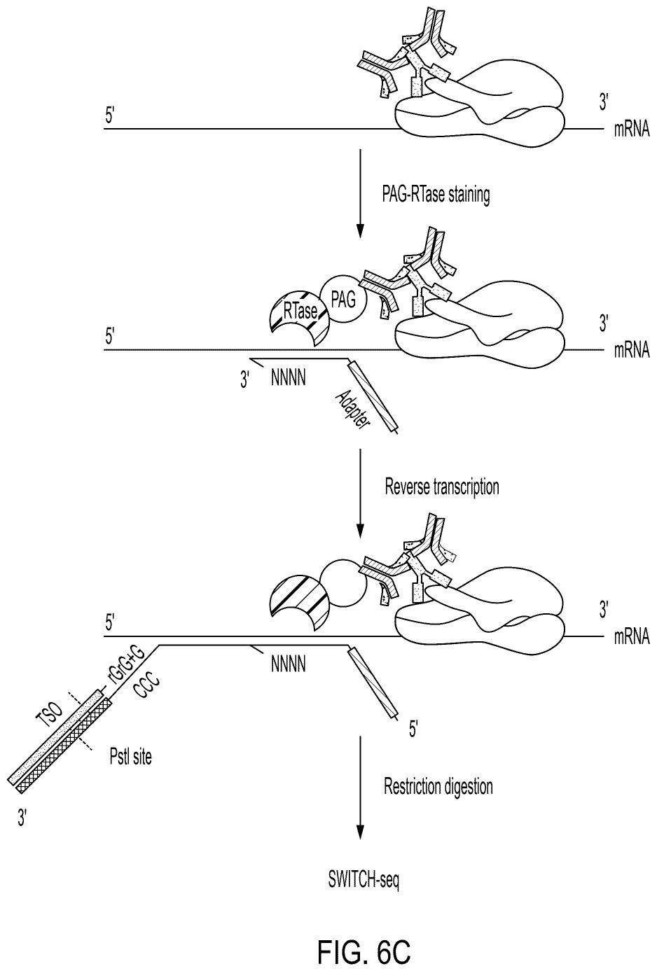

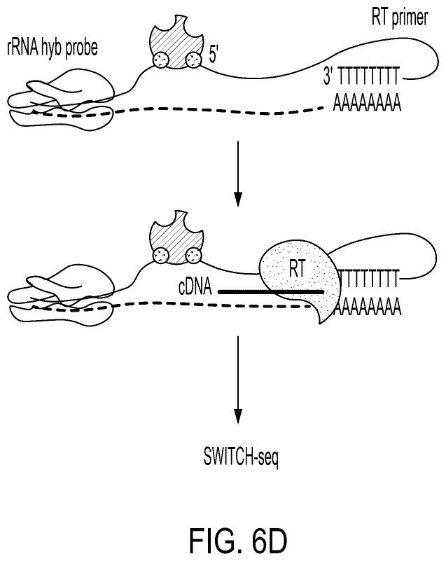

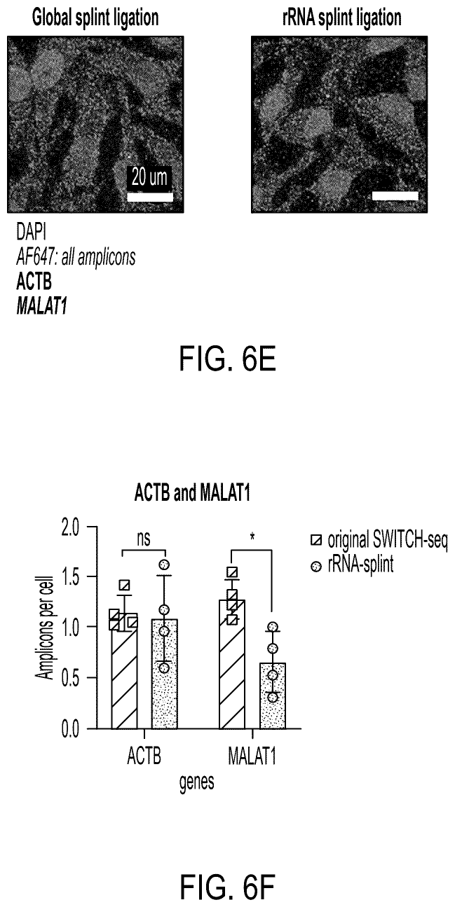

In some embodiments, the methods of the present disclosure are used to selectively profile RNAs that are bound to a ribosome. In some embodiments, the method further comprises contacting the cell with a splint probe. In some embodiments, the splint probe comprises a binding moiety, an oligonucleotide portion that is complementary to a ribosomal RNA (rRNA), an oligonucleotide portion that is complementary to the second oligonucleotide portion of a primer probe, and an oligonucleotide portion that is complementary to at least a portion of the sequence added to the 3′ end of the cDNA by the reverse transcriptase (see, e.g., FIG. 6A). In this way, the splint probe is capable of bringing the 5′ end and the 3′ end of each cDNA molecule produced in the methods provided herein in close proximity to one another to facilitate their ligation into a circular cDNA molecule only when a ribosome comprising an rRNA is bound to the RNA being profiled.

The splint probes of the present disclosure may comprise a polymerization blocker at their 3′ ends to prevent the splint probes from being used as a primer for reverse transcription in the methods provided herein. The polymerization blocker may be a small molecule or any other chemical modification that prevents a reverse transcriptase from adding a nucleotide to the 3′ end of the splint probe. In some embodiments, the polymerization blocker comprises an inverted nucleotide (e.g., an inverted thymine, uracil, adenine, guanine, or cytosine). In some embodiments, the polymerization blocker comprises an inverted thymine.

In some embodiments, the portion of the splint probe that is complementary to the sequence added to the 3′ end of the cDNA comprises a restriction endonuclease recognition site. In some embodiments, the restriction endonuclease recognition site is a AclI, HindIII, SspI, MluCI, PciI, AgeI, BfuAI, BspMI, SexAI, MluI, BceAI, HpyCH4IV, HpyCH4III, Bael, BsaXI, AflIII, SpeI, BsrI, BmrI, BgIII, AfeI, AluI, StuI, ScaI, ClaI, BspDI, PI-SceI, NsiI, AseI, SwaI, CspCI, MfeI, PaqCI, BssSI, BmgBI, PmlI, DraIII, AleI-v2, EcoP15I, PvuII, AlwNI, BtsIMutI, NdeI, NlaIII, FatI, MslI, XcmI, BstXI, PfIMI, BccI, NcoI, BseYI, FauI, SmaI, TspMI, XmaI, Nt.CviPII, AciI, SacII, BsrBI, MspI, HpaII, ScrFI, StyD4I, BsaJI, BslI, BtgI, NciI, AvrII, MnlI, BbvCI, Nt.BbvCI, Nb.BbvCI, SbfI, Bpu10I, Bsu36I, EcoNI, HpyAV, PspGI, BstNI, StyI, BcgI, PvuI, BstUI, EagI, RsrII, BsiEI, BsiWI, Esp3I, BsmBI-v2, Hpy99I, MspA1I, MspJI, SgrAI, BfaI, BspCNI, PaeR7I, XhoI, EarI, AcuI, PstI, BpmI, DdeI, SfcI, AflII, BpuEI, SmlI, AvaI, BsoBI, MboII, BbsI, XmnI, BsmI, Nb.BsmI, EcoRI, HgaI, AatII, ZraI, PflFI, Tth111I, PshAI, AhdI, DrdI, Eco53kI, SacI, BseRI, MlyI, PleI, Nt.BstNBI, HinfI, EcoRV, DpnII, Sau3 AI, MboI, DpnI, BsaBI, TfiI, BsrDI, Nb.BsrDI, BbvI, BtsI-v2, Nb.BtsI, BstAPI, SfaNI, SphI, SrfI, NmeAIII, NgoMIV, NaeI, BglI, AsiSI, BtgZI, HinP1I, HhaI, BssHII, NotI, Fnu4HI, Cac8I, MwoI, BmtI, NheI, BspQI, SapI, Nt.BspQI, BlpI, ApeKI, TseI, Bsp1286I, AlwI, BamHI, Nt.AlwI, BtsCI, FokI, HaeIII, FseI, SfiI, SfoI, PluTI, NarI, KasI, AscI, EciI, BsmFI, ApaI, PspOMI, Sau96I, NlaIV, KpnI, Acc65I, BsaI, HphI, BstEII, AvaII, BanI, BaeGI, BsaHI, BanII, RsaI, CviQI, BstZ17I, BciVI, SalI, BcoDI, BsmAI, Nt.BsmAI, ApaLI, BsgI, AccI, Hpy166II, Tsp45I, HpaI, PmeI, HincII, BsiHKAI, TspRI, ApoI, NspI, BsrFI-v2, BstYI, HaeII, CviKI-1, EcoO109I, PpuMI, I-CeuI, SnaBI, I-SceI, BspHI, BspEI, MmeI, TaqI-v2, NruI, Hpy188I, Hpy188III, XbaI, BclI, HpyCH4V, FspI, PI-PspI, MscI, BsrGI, MseI, PacI, PsiI-v2, BstBI, DraI, PspXI, BsaWI, BsaAI, or EaeI recognition site. In certain embodiments, the restriction endonuclease recognition site is a BstZ17I recognition site. In some embodiments, the step of contacting the cell with the RNase further comprises contacting the cell with a restriction endonuclease that cleaves the cDNA at the restriction endonuclease recognition site. This is useful, for example, for giving the cDNA a defined 3′ end.

In some embodiments, the binding moiety of the splint probe comprises a small molecule. In certain embodiments, the binding moiety comprises biotin. In some embodiments, the methods provided herein further comprise contacting the cell with a protein that binds to the binding moiety of the splint probe and performing a crosslinking reaction (e.g., reaction with bis-succinimide (PEG)₉ (BS(PEG)₉)). The splint probe will thereby be fixed in place, allowing only RNAs that are bound to a ribosome to be profiled. In some embodiments, the protein that binds to the binding moiety of the splint probe is streptavidin. In some embodiments, the binding moiety of the splint probe is an antigen, and the protein that binds to the binding moiety of the splint probe is an antibody.

In some embodiments, the splint probe used for profiling RNAs bound to a ribosome using the methods provided herein is 45-120, 50-115, 55-110, 60-115, 65-120, 70-115, 75-110, 80-105, or 85-100 nucleotides in length. In some embodiments, the splint probe is 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides in length. In some embodiments, the portion of the splint probe that is complementary to an rRNA is 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the oligonucleotide portion of the splint probe that is complementary to the second oligonucleotide portion of a primer probe is 20-40, 21-39, 22-38, 23-37, 24-36, 25-35, or 26-34 nucleotides in length. In some embodiments, the oligonucleotide portion of the splint probe that is complementary to the second oligonucleotide portion of a primer probe is 20, 21, 22, 23, 24, 25, 26, 27, 28, 29, 30, 31, 32, 33, 34, 35, 36, 37, 38, 39, or 40 nucleotides in length. In some embodiments, the portion of the splint probe that is complementary to the reverse complement of the TSO is 15-50 nucleotides in length. In some embodiments, the portion of the splint probe that is complementary to the reverse complement of the TSO is 21-42 nucleotides in length. In certain embodiments, the portion of the splint probe that is complementary to the reverse complement of the TSO is 21, 22, 23, 24, 25, 26, 27, 28, 29, 30, 31, 32, 33, 34, 35, 36, 37, 38, 39, 40, 41, or 42 nucleotides in length.

In some embodiments, the methods provided herein further comprise contacting the cell with a blocking probe. The blocking probe may be complementary to at least one portion of the splint probe, so that it can hybridize to the splint probe and prevent it from annealing to a primer probe before or while the primer probe is being used by a reverse transcriptase to prime reverse transcription. In some embodiments, the blocking probe comprises a first RNA portion complementary to the portion of the splint probe that is complementary to the second oligonucleotide portion of a primer probe and a second RNA portion that is complementary to the portion of the splint probe that is complementary to the sequence added to the 3′ end of the cDNA by the reverse transcriptase. Following production of cDNA from the primer probes, the blocking probes provided to the cell may then be degraded when RNase is provided to the cell in the method, allowing the splint probe to participate in circularization of the cDNA. In some embodiments, following the step of contacting the cell with an RNase, a 5′ portion and a 3′ portion of the cDNA produced from the primer probe anneal to the splint probe and are ligated together to produce the circular cDNA molecules. In some embodiments, the 5′ end and the 3′ end of each cDNA are ligated together by providing the cell with a DNA ligase.

The blocking probes of the present disclosure may comprise a polymerization blocker at their 3′ ends to prevent the blocking probes from being used as a primer for reverse transcription in the methods provided herein. The polymerization blocker may be a small molecule or any other chemical modification that prevents a reverse transcriptase from adding a nucleotide to the 3′ end of the splint probe. In some embodiments, the polymerization blocker comprises an inverted nucleotide (e.g., an inverted thymine, uracil, adenine, guanine, or cytosine). In some embodiments, the polymerization blocker comprises an inverted thymine.

In some embodiments, the blocking probe is 40-90, 45-85, 50-80, 55-75, or 60-70 nucleotides in length. In some embodiments, the blocking probe is 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 nucleotides in length. In some embodiments, the first RNA portion of the blocking probe is 20-40, 21-39, 22-38, 23-37, 24-36, 25-35, or 26-34 nucleotides in length. In some embodiments, the first RNA portion of the blocking probe is 20, 21, 22, 23, 24, 25, 26, 27, 28, 29, 30, 31, 32, 33, 34, 35, 36, 37, 38, 39, or 40 nucleotides in length. In some embodiments, the second RNA portion of the blocking probe is 15-50 nucleotides in length. In some embodiments, the second RNA portion of the blocking probe is 21-42 nucleotides in length. In some embodiments, the second RNA portion of the blocking probe is 21, 22, 23, 24, 25, 26, 27, 28, 29, 30, 31, 32, 33, 34, 35, 36, 37, 38, 39, 40, 41, or 42 nucleotides in length.

In some embodiments, the methods provided herein further comprise providing a fixer probe to the cell. In some embodiments, the fixer probe comprises a portion that is complementary to a portion of the primer probe and further comprises a binding moiety. The fixer probe may be useful for fixing the cDNA in place in the cell to prevent its movement prior to being amplified and imaged. In some embodiments, the binding moiety comprises a small molecule. In some embodiments, the binding moiety comprises biotin. In some embodiments, the method further comprises contacting the cell with a protein that binds to the binding moiety of the fixer probe and performing a crosslinking reaction to fix the fixer probe hybridized to the cDNA in place. In certain embodiments, the protein that binds to the binding moiety of the fixer probe is streptavidin. In some embodiments, the binding moiety of the fixer probe is an antigen, and the protein that binds the binding moiety of the fixer probe is an antibody.

In some embodiments, the primer probes used in the methods provided herein each comprise a binding moiety (e.g., at their 5′ end, or at an internal position on the primer probe that does not interfere with use of the 3′ end as a primer for reverse transcription). In certain embodiments, the binding moiety is attached to the 5′ end of the primer probes. The inclusion of a binding moiety on the primer probes may be useful, for example, for fixing the cDNA molecules in place once they have been polymerized from the primer probes, preventing them from migrating within the cell prior to being embedded within the hydrogel. In some embodiments, the method further comprises contacting the cell with a protein that binds to the binding moiety of the primer probes and performing a crosslinking reaction to fix the cDNA produced from the primer probes in place. In certain embodiments, the protein that binds to the binding moiety of the primer probes is streptavidin. In some embodiments, the binding moiety of the primer probes is an antigen, and the protein that binds to the binding moiety of the primer probes is an antibody.

In some embodiments, the step of performing rolling circle amplification to amplify the circular oligonucleotide to produce one or more concatenated amplicons further comprises providing nucleotide analogs modified with reactive chemical groups (e.g., amine modified nucleotides or any nucleotides comprising a nucleophile, such as 5-(3-aminoallyl)-dUTP). In some embodiments, the nucleotides modified with reactive chemical groups make up about 5%, about 6%, about 7%, about 8%, about 9%, or about 10% of the nucleotides used in the amplification reaction. For example, the step of performing rolling circle amplification to amplify the circular oligonucleotide to produce one or more concatenated amplicons may further comprise providing nucleotides comprising a nucleophile, including amine-modified nucleotides such as 5-(3-aminoallyl)-dUTP. During the amplification process, the amine-modified nucleotides are incorporated into the one or more concatenated amplicons as they are produced. The resulting amplicons are functionalized with primary amines, which can be further reacted with another compatible chemical moiety (e.g., N-hydroxysuccinimide) to facilitate the step of embedding the concatenated amplicons in the polymeric matrix. In some embodiments, the step of embedding the one or more concatenated amplicons in a polymeric matrix comprises reacting the amine-modified nucleotides of the one or more concatenated amplicons with a crosslinking agent (e.g., acrylic acid N-hydroxysuccinimide ester) and co-polymerizing the one or more concatenated amplicons and the polymer matrix.

A polymeric matrix is used in the methods described herein following rolling circle amplification to facilitate sequencing and imaging of the cDNAs produced by reverse transcription. The use of various polymeric matrices is contemplated by the present disclosure, and any polymeric matrix in which the one or more concatenated amplicons can be embedded is suitable for use in the methods described herein. In some embodiments, the polymeric matrix is a hydrogel (i.e., a network of crosslinked polymers that are hydrophilic). In some embodiments, the hydrogel is a polyvinyl alcohol hydrogel, a polyethylene glycol hydrogel, a polyacrylate hydrogel, or a polyacrylamide hydrogel. In certain embodiments, the hydrogel is a polyacrylamide hydrogel. Such a hydrogel may be prepared, for example, by incubating the sample in a buffer comprising acrylamide and bis-acrylamide, removing the buffer, and incubating the sample in a polymerization mixture (comprising, e.g., ammonium persulfate and tetramethylethylenediamine). Such reagents may also be provided in a kit, e.g., a kit for performing any of the methods described herein, or any of the kits described herein.

The methods disclosed herein also include a step of sequencing the concatenated amplicons, or a portion thereof, embedded in the polymeric matrix. In some embodiments, the step of sequencing comprises performing “sequencing with error-reduction by dynamic annealing and ligation” (SEDAL sequencing). SEDAL sequencing is described further in Wang, X. et al., Three-dimensional intact-tissue sequencing of single-cell transcriptional states. Science 2018, 361, 380, and International Patent Application Publication No. WO 2019/199579, published Oct. 17, 2019, each of which is incorporated herein by reference. In brief, oligonucleotide sequencing probes each comprising a detectable label (i.e., any label that can be used to visualize the location of the oligonucleotide sequencing probes, for example, through imaging) are provided to the cell. In certain embodiments, the detectable label is fluorescent (e.g., a fluorophore). In certain embodiments, the detectable label is a small molecule, such as an Alexa Fluor dye. In some embodiments, the oligonucleotide sequencing probes each comprise a random nucleic acid sequence. In certain embodiments, the random nucleic acid sequence is 5, 6, 7, 8, 9, 10, 11, or 12 nucleotides in length. In some embodiments, the oligonucleotide sequencing probes comprise oligonucleotides of the sequences NNNNNNAA, NNNNNNCC, NNNNNNGG, NNNNNNTT, NNNNNNAC, NNNNNNCA, NNNNNNGT, NNNNNNTG, NNNNNNAG, NNNNNNGA, NNNNNNCT, NNNNNNTC, NNNNNNAT, NNNNNNTA, NNNNNNGC, and/or NNNNNNCG, wherein each N is independently any nucleotide.

The oligonucleotide sequencing probes used in the methods described herein (e.g., as used in SEDAL sequencing) may be read out using any suitable imaging technique known in the art. For example, in embodiments where the oligonucleotide sequencing probes comprise a fluorophore, the fluorophore may be read out using imaging to sequence and identify each RNA. In some embodiments, imaging comprises fluorescent imaging. In certain embodiments, imaging comprises confocal microscopy. In certain embodiments, imaging comprises epifluorescence microscopy. In certain embodiments, two rounds of imaging are performed. In certain embodiments, three rounds of imaging are performed. In certain embodiments, four rounds of imaging are performed. In certain embodiments, five or more rounds of imaging are performed.

The use of any type of cell in the methods disclosed herein is contemplated by the present disclosure (e.g., any of the cell types described herein). In some embodiments, the cell is a mammalian cell. In certain embodiments, the cell is a human cell. The present disclosure also contemplates performing the methods for profiling RNA expression described herein on multiple cells simultaneously. In some embodiments, the method is performed on multiple cells of the same cell type. In some embodiments, the method is performed on multiple cells comprising cells of different cell types. In some embodiments, RNA expression is profiled in more than 10 cells, more than 20 cells, more than 50 cells, more than 10⁰ cells, more than 200 cells, more than 300 cells, more than 400 cells, more than 500 cells, or more than 1000 cells simultaneously. The cell types in which RNA expression may be profiled using the methods disclosed herein include, but are not limited to, stem cells, progenitor cells, neuronal cells, astrocytes, dendritic cells, endothelial cells, microglia, oligodendrocytes, muscle cells, myocardial cells, mesenchymal cells, epithelial cells, immune cells, hepatic cells, smooth and skeletal muscle cells, hematopoietic cells, lymphocytes, monocytes, neutrophils, macrophages, natural killer cells, mast cells, adipocytes, and neurons.

In certain embodiments, the cell or cells are present within an intact tissue (e.g., of any of the tissue types described herein). In certain embodiments, the intact tissue is a fixed tissue sample. In some embodiments, the intact tissue comprises cells of multiple cell types. In some embodiments, the tissue is epithelial tissue, connective tissue, muscular tissue, cardiac tissue, brain tissue, nervous tissue, tumor tissue, lymph node tissue, liver tissue, bone tissue, eye tissue, or ear tissue.

RNAs profiled in the methods described herein may be transcripts that have been expressed from the genomic DNA of the cell. In some embodiments, the RNAs of interest are messenger RNA (mRNA) and/or ribosomal RNA (rRNA). In some embodiments, the RNAs of interest comprise transcripts that have not yet been processed (e.g., pre-mRNA). In some embodiments, the RNAs of interest are transfer RNAs (tRNAs). The methods described herein may be used to profile expression of one RNA in a cell at a time, or of multiple RNAs simultaneously. In some embodiments, RNA expression in a cell, or in multiple cells, is profiled for more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 RNAs simultaneously.

In some embodiments, the methods for profiling RNA expression described herein may be combined with methods for profiling additional molecules within the cell. For example, methods for profiling other types of molecules (e.g., DNAs, proteins, carbohydrates, amino acids, metabolites, or lipids) may be combined with the methods described herein. In some embodiments, methods such as those described in International PCT Application Publication Nos. WO 2022/236011 (published Nov. 10, 2022), WO 2022/093940 (published Feb. 9, 2023), WO 2023/278409 (published Mar. 23, 2023), WO 2022/178274 (published Aug. 25, 2022), and WO 2023/018756 (published Feb. 16, 2023) may be combined with the methods described herein.

In some embodiments, the methods provided herein further comprise determining the cell type of the profiled cell by comparing the RNA expression profile of a cell to reference data comprising RNA expression profiles of various cell types. In some embodiments, the method further comprises overexpressing or knocking out one or more genes in the cell to determine whether the one or more genes are involved in expression of any RNAs in the cell. In some embodiments, the method further comprises repeating steps (a)-(h) in additional cells at different time points to profile RNA expression in the cell over time. In some embodiments, the methods further comprise examining how the profile of RNA expression in a cell or multiple cells is affected by an immune response within an intact tissue, or how the profile is affected due to proximity to a tumor.

### Methods for Diagnosing a Disease or Disorder in a Subject

In another aspect, the present disclosure provides methods for diagnosing a disease or disorder in a subject. For example, the methods for profiling RNA expression described herein may be performed on a cell or multiple cells (e.g., in an intact tissue) taken from a subject (e.g., a subject who is thought to have or is at risk of having a disease or disorder, or a subject who is healthy or thought to be healthy). The expression of various RNAs in the cell can then be compared to the expression of the same RNAs in a non-diseased cell or a cell from a non-diseased tissue sample (e.g., a cell from a healthy individual, or multiple cells from a population of healthy individuals). Any difference in the RNA expression profile of the cell (including of a single RNA or of multiple RNAs of interest, e.g., a specific disease signature) relative to one or more non-diseased cells may indicate that the subject has the disease or disorder.

In some embodiments, a method for diagnosing a disease or disorder in a subject comprises the steps of:
a) contacting a cell from a subject with a population of primer probes, wherein each primer probe in the population comprises a first oligonucleotide portion that is complementary to a portion of an RNA in the cell and a second oligonucleotide portion that is not complementary to the RNA;
b) contacting the cell with a reverse transcriptase, wherein the reverse transcriptase uses the first oligonucleotide portion of each of the primer probes to reverse transcribe the RNA to which each primer probe is hybridized, thereby producing a corresponding cDNA for each RNA;
c) contacting the cell with a template switching oligonucleotide (TSO), wherein a portion of the TSO is complementary to the 3′ end of each cDNA, and wherein the reverse transcriptase uses a portion of the TSO that is not complementary to the cDNA as a template to add the reverse complement sequence of the portion of the TSO to the 3′ end of the cDNA;
d) contacting the cell with an RNase, wherein the RNase digests all or substantially all of the RNA in the cell;
e) ligating the 5′ end and the 3′ end of each cDNA together to produce circular cDNA molecules;
f) performing rolling circle amplification to amplify the circular cDNA molecules, thereby producing a population of concatenated amplicons;
g) embedding the concatenated amplicons in a polymeric matrix; and
h) sequencing the concatenated amplicons, or a portion thereof, embedded in the polymeric matrix to determine the identity and location of RNAs in the cell;
wherein a difference in the profile of RNA expression in the cell relative to one or more non-diseased cells indicates that the subject has the disease or disorder.

In some embodiments, RNA expression in one or more non-diseased cells is profiled simultaneously alongside the cell taken from a subject using the methods disclosed herein as a control experiment. In some embodiments, the RNA expression profile of one or more non-diseased cells that is compared to expression in a diseased cell comprises reference data from when the method was performed on one or more non-diseased cells previously. Expression of a single RNA may be profiled in a cell to diagnose a disease or disorder in a subject using the methods disclosed herein, or expression of multiple different RNAs may be profiled in the cell simultaneously. In some embodiments, the difference in the profile of RNA expression comprises one or more mutations in an RNA (e.g., a single base substitution). In some embodiments, the difference in the profile of RNA expression comprises increased or decreased expression of one or more RNAs. In some embodiments, the difference in the profile of RNA expression comprises RNAs expressed at different levels in difference cell types or subcellular locations.

Diagnosis of any disease or disorder is contemplated by the methods described herein. In some embodiments, the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a neurological disease, or a cardiovascular disease.

In some embodiments, the cell is present in a tissue (e.g., epithelial tissue, connective tissue, muscular tissue, cardiac tissue, brain tissue, nervous tissue, or tumor tissue). In some embodiments, the tissue is a tissue sample from a subject. In some embodiments, the subject is a non-human experimental animal (e.g., a mouse, a rat, a non-human primate). In some embodiments, the subject is a domesticated animal. In some embodiments, the subject is a non-human primate. In some embodiments, the subject is a human. In some embodiments, the tissue sample comprises a fixed tissue sample. In certain embodiments, the tissue sample is a biopsy (e.g., bone, bone marrow, breast, gastrointestinal tract, lung, liver, pancreas, prostate, brain, nerve, renal, endometrial, cervical, lymph node, muscle, heart, or skin biopsy). In certain embodiments, the biopsy is a tumor biopsy.

### Methods for Treating a Disease or Disorder in a Subject

In another aspect, the present disclosure provides methods for treating a disease or disorder in a subject. For example, the methods for profiling RNA expression described herein may be performed in a cell (or in multiple cells, e.g., in an intact tissue) from a sample taken from a subject (e.g., a subject who is thought to have or is at risk of having a disease or disorder). The profile of RNA expression in the cell can then be compared to the RNA expression profile in a cell from a non-diseased tissue sample. A treatment for the disease or disorder may then be administered to the subject if any difference in the RNA expression profile to a non-diseased cell is observed.

In some embodiments, the present disclosure provides methods for treating a disease or disorder in a subject comprising the steps of:
a) contacting a cell taken from a subject with a population of primer probes, wherein each primer probe in the population comprises a first oligonucleotide portion that is complementary to a portion of an RNA in the cell and a second oligonucleotide portion that is not complementary to the RNA;
b) contacting the cell with a reverse transcriptase, wherein the reverse transcriptase uses the first oligonucleotide portion of each of the primer probes to reverse transcribe the RNA to which each primer probe is hybridized, thereby producing a corresponding cDNA for each RNA;
c) contacting the cell with a template switching oligonucleotide (TSO), wherein a portion of the TSO is complementary to the 3′ end of each cDNA, and wherein the reverse transcriptase uses a portion of the TSO that is not complementary to the cDNA as a template to add the reverse complement sequence of the portion of the TSO to the 3′ end of the cDNA;
d) contacting the cell with an RNase, wherein the RNase digests all or substantially all of the RNA in the cell;
e) ligating the 5′ end and the 3′ end of each cDNA together to produce circular cDNA molecules;
f) performing rolling circle amplification to amplify the circular cDNA molecules, thereby producing a population of concatenated amplicons;
g) embedding the concatenated amplicons in a polymeric matrix;
h) sequencing the concatenated amplicons, or a portion thereof, embedded in the polymeric matrix to determine the identity and location of RNAs in the cell; and
f) administering a treatment for the disease or disorder to the subject if a difference in the RNA expression profile of the cell relative to one or more non-diseased cells is observed.

In some embodiments, RNA expression in one or more non-diseased cells is profiled simultaneously using the methods disclosed herein as a control experiment. In some embodiments, the RNA expression profile of one or more non-diseased cells that is compared to the profile of a diseased cell comprises reference data from a time the method was performed on a non-diseased cell previously. In some embodiments, the difference in the profile of RNA expression comprises one or more mutations in an RNA (e.g., a single base substitution). In some embodiments, the difference in the profile of RNA expression comprises increased or decreased expression of one or more RNAs. In some embodiments, the difference in the profile of RNA expression comprises RNAs expressed at different levels in difference cell types or subcellular locations.

Any suitable treatment for a disease or disorder may be administered to the subject. In some embodiments, the treatment comprises administering a therapeutic agent. In some embodiments, the treatment comprises administering a prophylactic agent. In some embodiments, the treatment comprises surgery. In some embodiments, the treatment comprises imaging. In some embodiments, the treatment comprises performing further diagnostic methods. In some embodiments, the treatment comprises radiation therapy. In some embodiments, the treatment comprises a chance in diet or other lifestyle change. In some embodiments, the therapeutic agent is a small molecule, a protein, a peptide, a nucleic acid, a lipid, or a carbohydrate. In some embodiments, the therapeutic agent is a known drug and/or an FDA-approved drug. In certain embodiments, the therapeutic agent is a CRISPR-based treatment. In certain embodiments, the protein is an antibody. In certain embodiments, the protein is an antibody fragment or an antibody variant. In certain embodiments, the protein is a receptor, or a fragment or variant thereof. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), an antisense oligonucleotide (ASO), a DNA vector, or a viral vector.

Treatment of any disease or disorder is contemplated by the methods described herein. In some embodiments, the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a neurological disease, or a cardiovascular disease.

In some embodiments, the subject is a human. In some embodiments, the sample comprises a biological sample. In some embodiments, the sample comprises a tissue sample. In certain embodiments, the tissue sample is a biopsy (e.g., bone, bone marrow, breast, gastrointestinal tract, lung, liver, pancreas, prostate, brain, nerve, renal, endometrial, cervical, lymph node, muscle, or skin biopsy). In certain embodiments, the biopsy is a tumor biopsy. In certain embodiments, the biopsy is a solid tumor biopsy. In certain embodiments, the RNA expression profile of the biological sample informs prognostic decisions that guide therapies including but not limited to, pharmacological interventions for treating various conditions such as diabetes, psychiatric disorders, liver disease, kidney disease, blood disease, endocrine or exocrine disorders, heart disease, cancer therapies such as chemotherapy, targeted therapies, immunotherapy (e.g., checkpoint inhibition, CAR-T, cancer vaccines, etc.), metabolic disorders, or immune and autoimmune disorders.

### Methods of Screening for an Agent Capable of Modulating RNA Expression

In another aspect, the present disclosure provides methods for screening for an agent (e.g., a therapeutic agent, or any kind of stimulus such as a mechanical force, light, heat, electricity, etc.) capable of modulating RNA expression. For example, the methods for profiling RNA expression described herein may be performed in a cell (or in multiple cells, e.g., in an intact tissue) in the presence of one or more candidate agents. The expression of various RNAs in the cell (e.g., a normal cell, or a diseased cell) can then be compared to the expression of the same RNAs in a cell that was not exposed to the one or more candidate agents. Any difference in the RNA expression profile relative to the cell that was not exposed to the candidate agent(s) may indicate that expression of the RNAs is modulated by the candidate agent(s).

In some embodiments, the present disclosure provides methods for screening for an agent capable of modulating expression of one or more RNAs:
a) contacting a cell that is being treated with or has been treated with a candidate agent with a population of primer probes, wherein each primer probe in the population comprises a first oligonucleotide portion that is complementary to a portion of an RNA in the cell and a second oligonucleotide portion that is not complementary to the RNA;
b) contacting the cell with a reverse transcriptase, wherein the reverse transcriptase uses the first oligonucleotide portion of each of the primer probes to reverse transcribe the RNA to which each primer probe is hybridized, thereby producing a corresponding cDNA for each RNA;
c) contacting the cell with a template switching oligonucleotide (TSO), wherein a portion of the TSO is complementary to the 3′ end of each cDNA, and wherein the reverse transcriptase uses a portion of the TSO that is not complementary to the cDNA as a template to add the reverse complement sequence of the portion of the TSO to the 3′ end of the cDNA;
d) contacting the cell with an RNase, wherein the RNase digests all or substantially all of the RNA in the cell;
e) ligating the 5′ end and the 3′ end of each cDNA together to produce circular cDNA molecules;
f) performing rolling circle amplification to amplify the circular cDNA molecules, thereby producing a population of concatenated amplicons;
g) embedding the concatenated amplicons in a polymeric matrix; and
h) sequencing the concatenated amplicons, or a portion thereof, embedded in the polymeric matrix to determine the identity and location of RNAs in the cell;
wherein a difference in the profile of RNA expression in the cell in the presence of the candidate agent relative to in the absence of the candidate agent indicates that the candidate agent modulates expression of one or more RNAs.

In some embodiments, the difference in the profile of RNA expression comprises one or more mutations in an RNA (e.g., a single base substitution). In some embodiments, the difference in the profile of RNA expression comprises increased or decreased expression of one or more RNAs. In some embodiments, the difference in the profile of RNA expression comprises RNAs expressed at different levels in difference cell types or subcellular locations.

In some embodiments, the candidate agent is a small molecule, a protein, a peptide, a nucleic acid, a lipid, or a carbohydrate. In some embodiments, the candidate agent comprises a known drug or an FDA-approved drug. In certain embodiments, the candidate agent is a CRISPR-based treatment. In certain embodiments, the protein is an antibody. In certain embodiments, the protein is an antibody fragment or an antibody variant. In certain embodiments, the protein is a receptor. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), an antisense oligonucleotide (ASO), a DNA vector, or a viral vector. In some embodiments, multiple candidate agents are provided as a screening library. Any candidate agent may be screened using the methods described herein. In particular, any candidate agents thought to be capable of modulating RNA expression in a desired manner may be screened using the methods described herein.

In some embodiments, modulation of RNA expression by the candidate agent is associated with reducing, relieving, or eliminating the symptoms of a disease or disorder, or preventing the development or progression of the disease or disorder. In some embodiments, the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a neurological disease, or a cardiovascular disease.

### Oligonucleotides

The present disclosure also provides oligonucleotide for use in the methods and systems for profiling RNA expression described herein. In one aspect, the present disclosure provides sets of oligonucleotides comprising: a primer probe comprising a first oligonucleotide portion that is complementary to a portion of an RNA in a cell and a second oligonucleotide portion that is not complementary to the RNA; and a template switching oligonucleotide (TSO).

The primer probes are capable of priming reverse transcription of RNAs in a cell. In some embodiments, each primer probe comprises the structure 5′-[second oligonucleotide portion not complementary to RNA]-[first oligonucleotide portion complementary to RNA]-3′, wherein]-[represents an optional linker (e.g., a nucleotide linker). In some embodiments,]-[represents a direct linkage between the two portions of the primer probe (i.e., a phosphodiester bond). In some embodiments, the first oligonucleotide portion of the primer probes comprises DNA. In some embodiments, the second oligonucleotide portion of the primer probes comprises DNA. In certain embodiments, both the first and the second oligonucleotide portions of the primer probes comprise DNA. In some embodiments, the first oligonucleotide portion of the primer probes comprises RNA. In some embodiments, the second oligonucleotide portion of the primer probes comprises RNA. In certain embodiments, both the first and the second oligonucleotide portions of the primer probes comprise RNA. In some embodiments, the primer probes comprise modified oligonucleotides or oligonucleotide analogs.

In some embodiments, the primer probes provided herein may be used to profile RNA expression in a cell in an untargeted manner. In such embodiments, the first oligonucleotide portion of each of the primer probes comprises an unknown sequence (i.e., a sequence of random nucleotides). The unknown sequence of each primer probe is thus capable of hybridizing to a random RNA sequence in a cell, thereby allowing for untargeted reverse transcription of said RNA sequence. In some embodiments, the first oligonucleotide portion of each of the primer probes comprises a random sequence of 5-12, 6-11, 7-10, or 8-9 nucleotides in length. In some embodiments, the first oligonucleotide portion of each of the primer probes comprises a random sequence of 5, 6, 7, 8, 9, 10, 11, or 12 nucleotides in length. In certain embodiments, the first oligonucleotide portion of each of the primer probes comprises a random sequence of 8 nucleotides in length.

In some embodiments, the primer probes provided herein may be used to profile RNA expression in a targeted manner. For example, the primer probes may be utilized to profile expression of RNAs with one or more mutations of interest (e.g., rRNAs comprising single base variants). In such embodiments, the first oligonucleotide portion of each of the primer probes comprises a known sequence. In some embodiments, the known sequence is complementary to a non-variable region of an RNA. In certain embodiments, the known sequence is complementary to a non-variable region of an rRNA. In some embodiments, the known sequence comprises a poly dT sequence. In some embodiments, the first oligonucleotide portion of each of the primer probes comprises a known sequence of 8-40, 9-39, 10-38, 11-37, 12-36, 13-35, 14-34, 15-33, 16-32, 17-31, 18-30, 19-29, 20-28, 21-27, 22-26, or 23-25 nucleotides in length. In some embodiments, the first oligonucleotide portion of each of the primer probes comprises a known sequence of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the first oligonucleotide portion of each of the primer probes comprises a known sequence with a melting temperature of 50-65° C.

In some embodiments, the second oligonucleotide portion of each primer probe comprises a known sequence. For example, the second oligonucleotide portion of each primer probe may comprise a known sequence at the 5′ end in order to facilitate circularization of cDNAs produced when using the primer probes to prime reverse transcription as described herein. In some embodiments, the second oligonucleotide portion of each primer probe is 20-40, 21-39, 22-38, 23-37, 24-36, 25-35, or 26-34 nucleotides in length. In some embodiments, the second oligonucleotide portion of each primer probe is 20, 21, 22, 23, 24, 25, 26, 27, 28, 29, 30, 31, 32, 33, 34, 35, 36, 37, 38, 39, or 40 nucleotides in length. In certain embodiments, the second oligonucleotide portion of each primer probe is 27 nucleotides in length. In some embodiments, the second oligonucleotide portion of each of the primer probes comprises a sequence at least 90%, at least 95%, at least 96%, at least 97%, at least 98%, or at least 99% identical to the sequence TATACACTAAAGATAGGATCCACT (SEQ ID NO: 1). In certain embodiments, the second oligonucleotide portion of each of the primer probes comprises the sequence TATACACTAAAGATAGGATCCACT (SEQ ID NO: 1).

In some embodiments, the second oligonucleotide portion of each of the primer probes comprises a secondary structural motif at the 5′ end. In some embodiments, the secondary structural motif is a hairpin sequence. In certain embodiments, the hairpin sequence comprises a sequence at least 90%, at least 95%, at least 96%, at least 97%, at least 98%, or at least 99% identical to the sequence CACCGAGACTGCGAGTCACACATACACTAAAGCTTGGGACAGACTACCTCTCTC GCAGTCTCGGT (SEQ ID NO: 2). In certain embodiments, the hairpin sequence comprises the sequence CACCGAGACTGCGAGTCACACATACACTAAAGCTTGGGACAGACTACCTCTCTC GCAGTCTCGGT (SEQ ID NO: 2).

The sets of oligonucleotides described herein also comprise a template switching oligonucleotide (TSO). A portion of the TSO provided herein is complementary to the 3′ end of a cDNA produced using the methods described herein. An additional portion of the TSO is not complementary to the cDNA and is used as a template to add an additional sequence to the 3′ end of the cDNA. In some embodiments, the portion of the TSO that is complementary to the 3′ end of each cDNA is complementary to additional non-templated nucleotides added by a reverse transcriptase at the 3′ end of the cDNA (e.g., a sequence of untemplated deoxycytidines). In some embodiments, the portion of the TSO that is complementary to the 3′end of a cDNA comprises one or more guanosines and/or deoxyguanosines. In some embodiments, the portion of the TSO that is complementary to the 3′ end of a cDNA comprises two, three, four, or five guanosines and/or deoxyguanosines. In certain embodiments, the portion of the TSO that is complementary to the 3′ end of a cDNA comprises three guanosines and/or deoxyguanosines. In some embodiments, the TSO comprises DNA. In some embodiments, the TSO comprises RNA. In certain embodiments, the TSO comprises a hybrid of DNA and RNA. In some embodiments, the TSO comprises modified nucleotides or nucleotide analogs. In some embodiments, the TSO comprises a secondary structural motif at its 3′ end. In certain embodiments, the secondary structural motif is a hairpin sequence. In some embodiments, the TSO is 15-50 nucleotides in length. In some embodiments, the TSO is 21-42 nucleotides in length. In certain embodiments, the TSO is 21, 22, 23, 24, 25, 26, 27, 28, 29, 30, 31, 32, 33, 34, 35, 36, 37, 38, 39, 40, 41, or 42 nucleotides in length.

In some embodiments, the TSO comprises a sequence at least 90%, at least 95%, at least 96%, at least 97%, at least 98%, or at least 99% identical to any one of the sequences: TACCAACGCAGAGTACATrGrGG (SEQ ID NO: 4), rUrArCrCrArArCrGrCrArGrArGrUrArCrArUrGrGG (SEQ ID NO: 5), rUrArCrCrArACGCrArGrArGrUrACATrGrGG (SEQ ID NO: 6), and AACCGAGACTGCGAGAAATACCCNNNNNCATCCCTGCCAAGCTTCTAACTCGCA GTCTCGGTTTCGTAGACTAAGATrGrGG (SEQ ID NO: 7). In certain embodiments, the TSO comprises the sequence: TACCAACGCAGAGTACATIGrGG (SEQ ID NO: 4), rUrArCrCrArArCrGrCrArGrArGrUrArCrArUrGrGG (SEQ ID NO: 5), rUrArCrCrArACGCrArGrArGrUrACATrGrGG (SEQ ID NO: 6), or AACCGAGACTGCGAGAAATACCCNNNNNCATCCCTGCCAAGCTTCTAACTCGCA GTCTCGGTTTCGTAGACTAAGATrGrGG (SEQ ID NO: 7).

In some embodiments, the set of oligonucleotides further comprises a circularization probe. The circularization probes provided herein comprise a portion that is complementary to the second oligonucleotide portion of the primer probe, and another portion that is complementary to the reverse complement of a portion of the TSO. In this way, the circularization probes are capable of bringing the 5′ end and the 3′ end of each cDNA molecule produced in the methods provided herein in close proximity to one another to facilitate their ligation into a circular molecule.

In some embodiments, the circularization probe is 30-70, 31-69, 32-68, 33-67, 34-66, 35-65, 36-64, 37-63, 38-62, 39-61, 40-60, 41-59, 42-58, 43-57, 44-56, or 45-55 nucleotides in length. In some embodiments, the circularization probe is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length. In certain embodiments, the circularization probe is 48 nucleotides in length. In some embodiments, the circularization probe comprises a sequence at least 90%, at least 95%, at least 96%, at least 97%, at least 98%, or at least 99% identical to the sequence AGTGGATCCTATCTTTAGTGTATATACCAACGCAGAGTACAT (SEQ ID NO: 8). In certain embodiments, the circularization probe comprises the sequence AGTGGATCCTATCTTTAGTGTATATACCAACGCAGAGTACAT (SEQ ID NO: 8).

In some embodiments, the present disclosure provides additional probes that may be useful for utilizing the methods provided herein to profile RNAs that are bound to a ribosome. In some embodiments, the set of oligonucleotides further comprises a splint probe. In some embodiments, the splint probe comprises a binding moiety, an oligonucleotide portion that is complementary to an rRNA, an oligonucleotide portion that is complementary to the second oligonucleotide portion of a primer probe, and an oligonucleotide portion that is complementary to at least a portion of the reverse complement of the TSO. In this way, the splint probe is capable of bringing the 5′ end and the 3′ end of each cDNA molecule produced in the methods provided herein in close proximity to one another to facilitate their ligation into a circular cDNA molecule only when a ribosome comprising an rRNA is bound to the RNA being profiled. In some embodiments, the binding moiety comprises a small molecule. In certain embodiments, the binding moiety comprises biotin.

In some embodiments, the splint probe is 45-120, 50-115, 55-110, 60-115, 65-120, 70-115, 75-110, 80-105, or 85-100 nucleotides in length. In some embodiments, the splint probe is 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides in length. In some embodiments, the portion of the splint probe that is complementary to an rRNA is 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the oligonucleotide portion of the splint probe that is complementary to the second oligonucleotide portion of a primer probe is 20-40, 21-39, 22-38, 23-37, 24-36, 25-35, or 26-34 nucleotides in length. In some embodiments, the oligonucleotide portion of the splint probe that is complementary to the second oligonucleotide portion of a primer probe is 20, 21, 22, 23, 24, 25, 26, 27, 28, 29, 30, 31, 32, 33, 34, 35, 36, 37, 38, 39, or 40 nucleotides in length. In some embodiments, the portion of the splint probe that is complementary to the reverse complement of the TSO is 15-50 nucleotides in length. In some embodiments, the portion of the splint probe that is complementary to the reverse complement of the TSO is 21-42 nucleotides in length. In certain embodiments, the portion of the splint probe that is complementary to the reverse complement of the TSO is 21, 22, 23, 24, 25, 26, 27, 28, 29, 30, 31, 32, 33, 34, 35, 36, 37, 38, 39, 40, 41, or 42 nucleotides in length.

In some embodiments, the set of oligonucleotides further comprises a blocking probe. In some embodiments, the blocking probe comprises a first RNA portion complementary to the portion of the splint probe that is complementary to the second oligonucleotide portion of a primer probe and a second RNA portion that is complementary to the portion of the splint probe that is complementary to the reverse complement of the TSO. The blocking probe may be used to prevent the splint probe from annealing to a primer probe before or while the primer probe is being used to produce cDNA in the methods described herein. The blocking probe may then be degraded when RNase is provided to the cell in the method, allowing the splint probe to participate in circularization of the cDNA.

In some embodiments, the blocking probe is 40-90, 45-85, 50-80, 55-75, or 60-70 nucleotides in length. In some embodiments, the blocking probe is 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 nucleotides in length. In some embodiments, the first RNA portion of the blocking probe is 20-40, 21-39, 22-38, 23-37, 24-36, 25-35, or 26-34 nucleotides in length. In some embodiments, the first RNA portion of the blocking probe is 20, 21, 22, 23, 24, 25, 26, 27, 28, 29, 30, 31, 32, 33, 34, 35, 36, 37, 38, 39, or 40 nucleotides in length. In some embodiments, the second RNA portion of the blocking probe is 15-50 nucleotides in length. In some embodiments, the second RNA portion of the blocking probe is 21-42 nucleotides in length. In some embodiments, the second RNA portion of the blocking probe is 21, 22, 23, 24, 25, 26, 27, 28, 29, 30, 31, 32, 33, 34, 35, 36, 37, 38, 39, 40, 41, or 42 nucleotides in length.

In some embodiments, the set of oligonucleotide probes further comprises a fixer probe. In some embodiments, the fixer probe comprises a portion that is complementary to a portion of the primer probe and further comprises a binding moiety. The fixer probe may be useful for fixing the cDNA in place in the cell to prevent its movement prior to being amplified and imaged. In some embodiments, the binding moiety comprises a small molecule. In some embodiments, the binding moiety comprises biotin.

All of the oligonucleotides described herein may optionally have spacers or linkers of various nucleotide lengths in between each of the recited portions or components, or the portions or components of the oligonucleotides may be joined directly to one another (i.e., by a phosphodiester bond). All of the oligonucleotides described herein may comprise standard nucleotides, or some of the standard nucleotides may be substituted for any modified nucleotides known in the art.

In some embodiments, the present disclosure provides a plurality of oligonucleotides comprising multiple sets of oligonucleotides as described herein. In certain embodiments, each set of oligonucleotides in the plurality comprises a primer probe that is complementary to a different RNA in a cell. In some embodiments, the plurality of oligonucleotides comprises more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of oligonucleotides.

### Kits

Also provided by the disclosure are kits. In one aspect, the kits provided may comprise one or more of the oligonucleotides described herein. In some embodiments, the kits comprise any of the sets of oligonucleotides or pluralities of oligonucleotides described herein. In certain embodiments, the kits comprise more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of oligonucleotides or pluralities of oligonucleotides. In some embodiments, the kits may further comprise a container (e.g., a vial, ampule, bottle, and/or dispenser package, or other suitable container). The kits may also comprise cells for performing control experiments. In some embodiments, the kits may further comprise other reagents for performing the methods disclosed herein (e.g., enzymes such as a ligase, a polymerase (e.g., a DNA polymerase and/or a reverse transcriptase), and/or an RNase, nucleotides comprising a nucleophile (e.g., amine-modified nucleotides) as described herein, buffers, and/or reagents and monomers for making a polymeric matrix (e.g., a polyacrylamide matrix)).

In some embodiments, the kits are useful for profiling RNA expression in a cell. In some embodiments, the kits are useful for profiling RNA expression in a cell in an untargeted manner. In some embodiments, the kits are useful for profiling RNA expression in a cell in a targeted manner. In some embodiments, the kits are useful for diagnosing a disease in a subject. In some embodiments, the kits are useful for screening for an agent capable of modulating expression of one or more RNAs. In some embodiments, the kits are useful for diagnosing a disease or disorder in a subject. In some embodiments, the kits are useful for treating a disease or disorder in a subject. In certain embodiments, a kit described herein further includes instructions for using the kit.

### Systems

In one aspect, the present disclosure provides systems for profiling RNA expression in a cell. In some embodiments, such a system comprises:

a) a cell, tissue, or biological sample;

b) one or more primer probes, wherein each primer probe comprises a first oligonucleotide portion that is complementary to a portion of an RNA in the cell and a second oligonucleotide portion that is not complementary to the RNA;

c) a reverse transcriptase; and

d) a template switching oligonucleotide (TSO).

Any of the oligonucleotides (i.e., the sets of oligonucleotides and pluralities of oligonucleotides) described herein may be used in the systems contemplated by the present disclosure. In some embodiments, a system further comprises a microscope. In some embodiments, a system further comprises a computer. In certain embodiments, a system further comprises software running on the computer (e.g., software for viewing or processing the images observed on or captured with a microscope). In certain embodiments, a system further comprises a liquid handling system. In some embodiments, the systems further comprise one or more enzymes. In certain embodiments, the systems further comprise an RNase. In certain embodiments, the systems further comprise a DNA ligase. In certain embodiments, the systems further comprise a DNA polymerase. In some embodiments, the systems further comprise additional reagents (e.g., dyes, stains, antibodies, etc.). In certain embodiments, the systems further comprise nucleotides comprising a nucleophile (e.g., amine-modified nucleotides). In certain embodiments, the systems further comprise one or more buffers. In certain embodiments, the systems further comprise reagents and monomers for preparing a polymeric matrix (e.g., a polyacrylamide matrix)).

### EXAMPLES

### Example 1: SWITCH-Seq: Template-Switching-Based Multiplexed In Situ RNA Sequencing Design and Validation of SWITCH-Seq

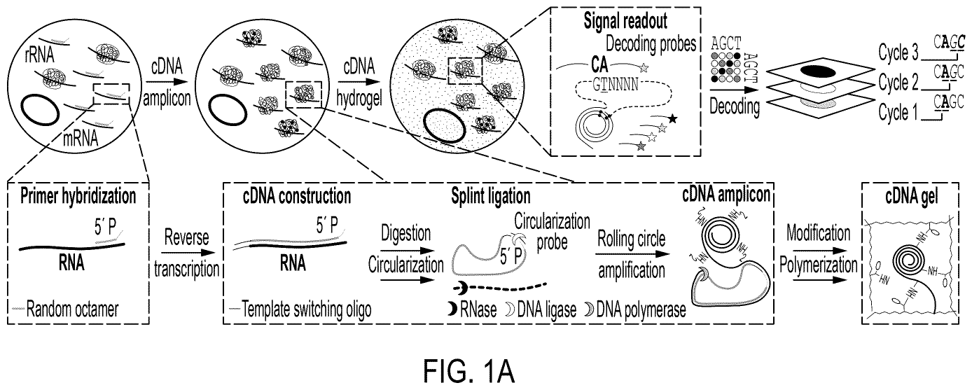

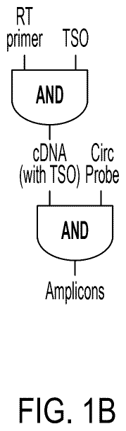

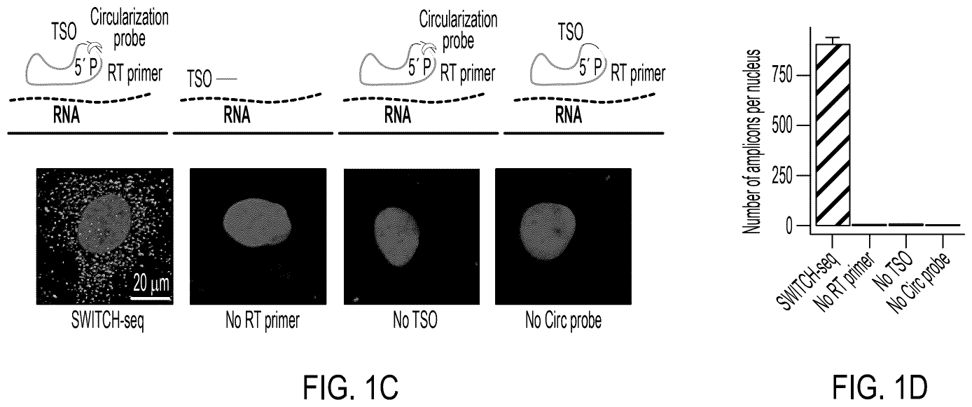

The process of SWITCH-seq begins with performing reverse transcription (RT) on fixed cells or tissue slices in situ, wherein a known sequence of choice is integrated into the 3′ end of cDNA via template switching (FIG. 1A, Methods). In the untargeted version of SWITCH-seq, random octamers, complemented by a 5′ flanking sequence, serve as the RT primer. As the reverse transcriptase (which has inherent terminal transferase activity) approaches the end of the RNA template, it appends a few additional nucleotides, primarily deoxycytidines, to the nascent cDNA's 3′ end13,14. These additional non-templated nucleotides can anneal to the template-switching oligonucleotide (TSO). This annealing occurrence prompts the reverse transcriptase to transition its templating source from the RNA to the TSO. The resultant cDNA bears a sequence complementary to the TSO at its 3′ end, a feature essential for subsequent circularization processes. To retain cDNA fragments and mitigate their diffusion, aminoallyl-dUTP was incorporated into the cDNA during RT. Subsequently, the cDNA fragments were cross-linked using BS(PEG)₉.

Upon completion of RT, residual RNA was enzymatically digested, yielding single-stranded cDNA poised for circularization. With the known 5′ end (5′ flanking sequence on the RT primer) and the 3′ end (complementary to the TSO sequence) of the cDNA, a circularization probe was employed. This probe hybridizes to the cDNA and serves as a splint to aid the circularization process. Furthermore, the same probe functions as the primer for in situ amplification of the circularized cDNA through rolling circle amplification (RCA), leading to the formation of cDNA nanoballs, or amplicons. Collectively, the methodology described herein ensures the selective amplification of RNA sequences that have been reverse transcribed with the integration of the TSO at the 3′ end and then circularized (FIGS. 1B-1D).

To facilitate the integration of cDNA amplicons within a tissue-hydrogel network, aminoallyl-dUTP was again introduced into the RCA procedure. This aminoallyl-dUTP subsequently underwent a reaction with acrylic acid N-hydroxysuccinimide esters. Thereafter, through copolymerization with acrylamide monomers, a distinct hydrogel-DNA amplicon composite was formed. The RNA profile was elucidated using SEDAL (sequencing with error-reduction by dynamic annealing and ligation)².

SWITCH-Seq Discerns Single-Base Variations in rRNA

An inherent advantage of untargeted in situ sequencing is its capability to discern spatially discrete sequence variants. Recent studies have utilized long-read sequencing on rDNA and rRNA within actively translating ribosomes, unearthing hundreds of variants. Notably, several highly abundant variants were identified in the expansion segments (ESs) regions, namely es15l, es27l, and es39l, as well as one non-ES area, helix 28S: h1115.

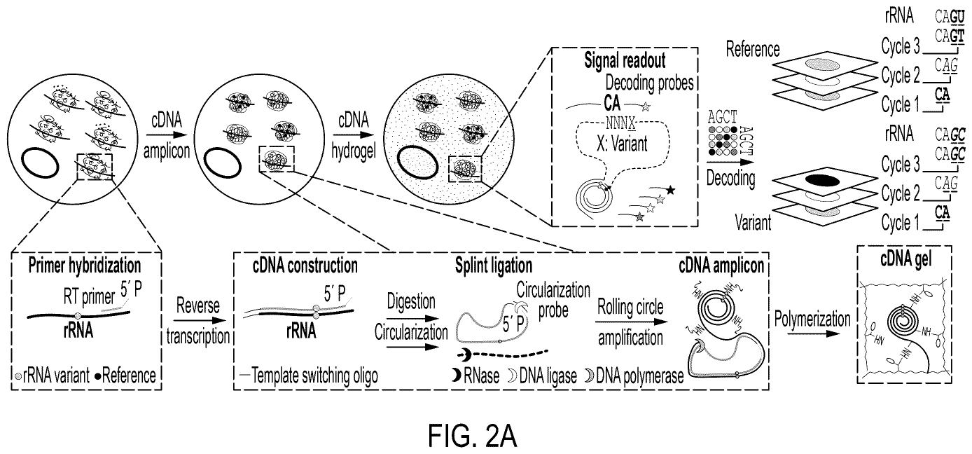

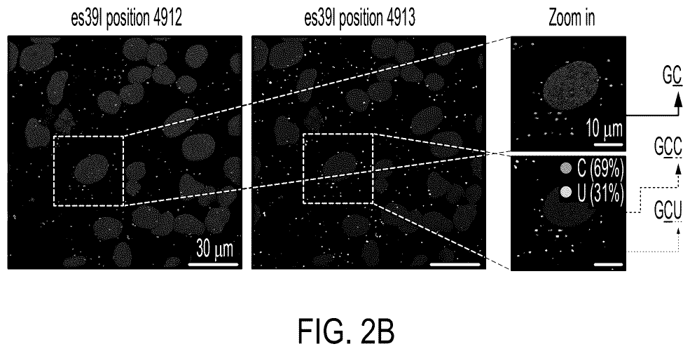

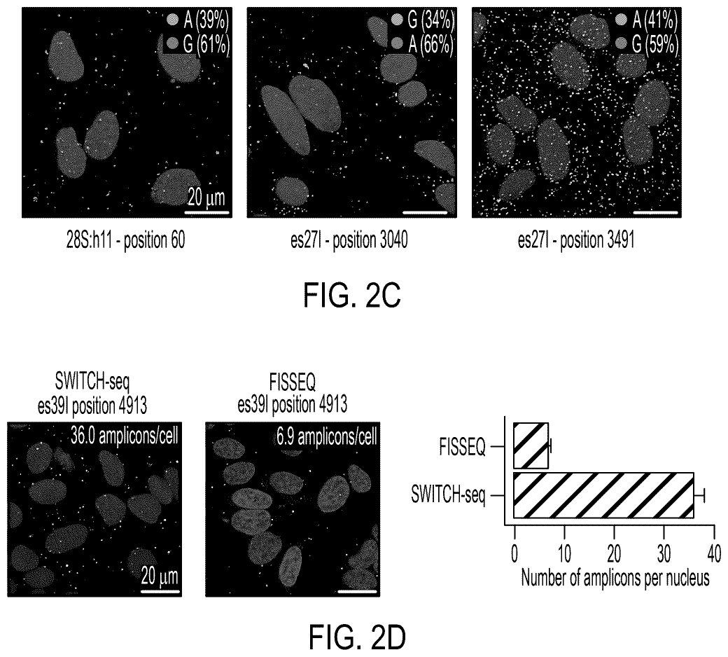

For these highly abundant variants, a strong correlation between the frequency of a variant's occurrence among rDNA copies and its expression levels in rRNA indicates both the authenticity of these sequence variants and their likely co-expression within individual cells. To explore this hypothesis, SWITCH-seq was tailored to specifically target designated rRNA regions, enabling visualization of variant ribosomes in Hela cells (FIG. 2A). Diverging from the untargeted version, instead of using a random octamer for the RT primer, an RT primer was designed that specifically targets the consistent, non-variable regions located downstream of the selected rRNA variant regions. Multiple rounds of imaging were conducted to confirm the two bases before the variants, and the consecutive base, where the variants were expected, was subsequently sequenced (FIG. 2B). As predicted, both the reference and alternate alleles were observed (FIGS. 2B-2C). Notably, the method demonstrated five times higher efficiency in detecting rRNA variants than FISSEQ (FIG. 2D). Furthermore, the frequencies of the reference and variant alleles corresponded with sequencing results. It was concluded that rRNA variants observed at high frequency are indeed co-expressed in individual cells that can be visualized at single-cell resolution.

### Optimization of SWITCH-Seq

In the course of developing SWITCH-seq, meticulous optimization across various aspects of the methodology was undertaken. A primary focus was the structure of the TSO. Conventionally, a TSO is composed of a DNA oligonucleotide sequence that carries three riboguanosines (rGrGrG) at its 3′ terminus, or alternatively, the 3′ most rG might be substituted with a locked nucleic acid base (rGrG+G)¹⁴. This sequence design leverages the complementarity between the successive rG bases and the 3′ dC extension of the cDNA molecule, facilitating the subsequent template-switching process. However, a predicament arises when DNA serves as the material for the TSO oligo: it remains resistant to RNase digestion. Consequently, this TSO persists in hybridizing to the 3′ terminus of the cDNA. This interaction could jeopardize the cDNA's circularization, given that the circularization probe also seeks to bind to the cDNA's 3′ end. To address this, the potential of RNA as an alternative material for the TSO was evaluated (FIG. 3A). Furthermore, a DNA/RNA hybrid was assessed for its TSO capabilities (FIG. 3A), considering that upon RNA digestion, the residual DNA may lack the requisite melting temperature for annealing to the cDNA at room temperature. These investigations revealed that both RNA and the DNA/RNA hybrid TSOs augmented the efficiency by approximately 50%.

The incorporation of RT primers and TSOs, both designed with hairpin structures, was also explored (FIG. 3B). After template switching, this approach yielded cDNAs with hairpin configurations at both the 5′ and 3′ ends. Following this, an RNase digestion step was initiated to remove any residual RNA. Phusion® High-Fidelity DNA Polymerase—known for its superior efficiency in gap-filling with padlock probes—was then employed for the gap-filling task₃. The ensuing ligation process achieved cDNA circularization. The subsequent procedures aligned with the conventional SWITCH-seq method: rolling circle amplification produced the cDNA amplicons, which were subsequently embedded into the hydrogel for cyclic imaging. However, the results pointed to diminished efficiency when using hairpin-structured TSOs (FIG. 3C). This observation was validated both in situ in HeLa cells and in vitro through qPCR analyses (FIG. 3C).

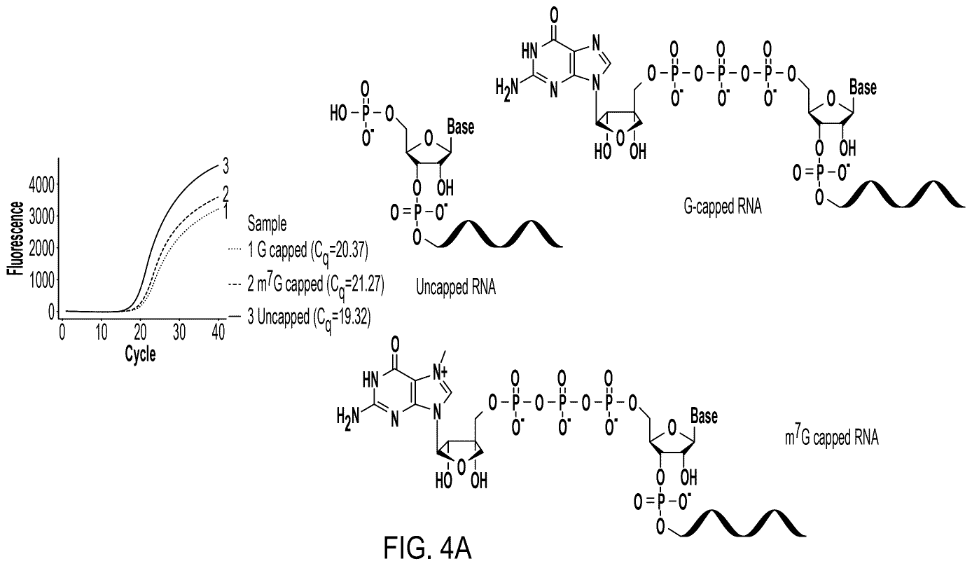

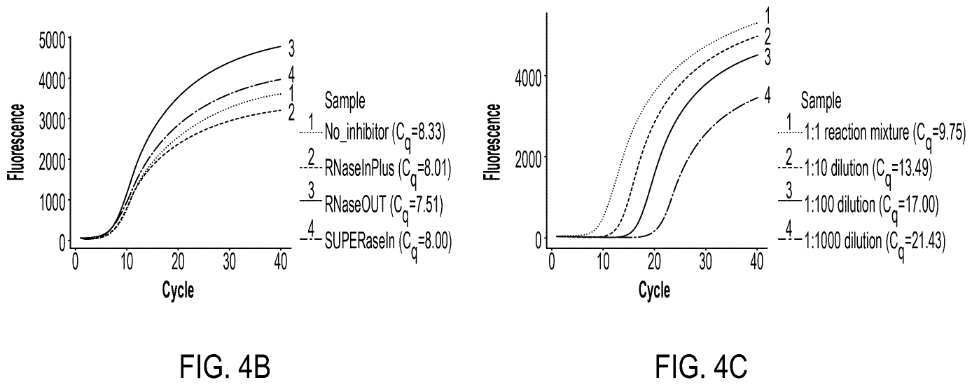

In pursuit of optimizing template switching efficiency, the potential influence of chemical capping at the 5′ end of the RNA16 was evaluated (FIG. 4A). Specifically, 5′-phosphate RNAs (uncapped), N7-methylguanosine capped (m7G-capped) RNAs, and guanosine-capped (G-capped) RNAs were compared in template switching experiments. However, qPCR analyses revealed that such chemical modifications did not enhance the template switching efficiency.

In light of reports suggesting that SUPERnaseIn™ may impede the activity of reverse transcriptase, various RNase inhibitors were systemically evaluated (FIG. 4B). Through in vitro qPCR analyses, the template switching efficiency when using RNaseIn™ Plus, RNaseOUT™, SUPERnaseIn™, and no inhibitor was examined. Based on these findings, RNaseOUT™ was selected as the optimal RNase inhibitor for the protocol.

For in vitro qPCR evaluations, a linear DNA TSO was employed unless stated otherwise. The qPCR primers were designed to target both the RT primer and the TSO. The efficiency of the qPCR was ascertained through serial dilution, yielding positive results (FIG. 4C).

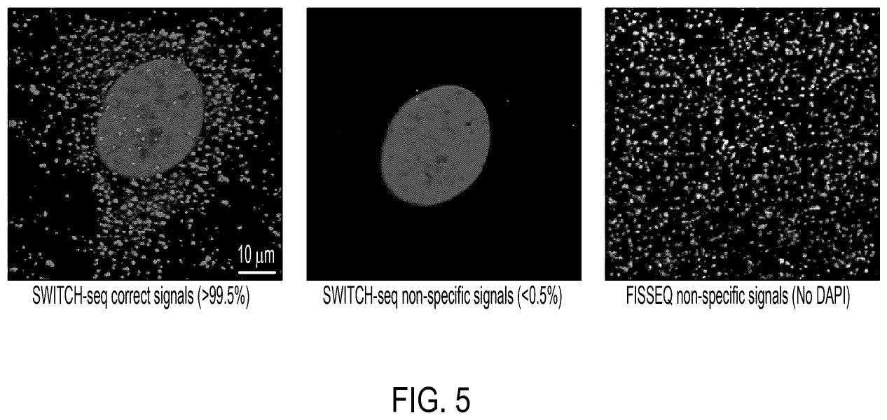

During work with FISSEQ when mapping rRNA variants, significant challenges related to primer self-circularization were observed, resulting in an excess of amplicons in areas absent of cells (FIG. 5). This observation prompted a thorough evaluation of specificity within the developed method. For this purpose, the reading sequence was annealed to the 5′ end of the RT primer. This reading sequence allowed identification of the terminal base of cDNA, anticipated to be “A,” as represented by the red channel in raw image reads. The additional three channels, corresponding to “T,” “G,” and “C,” were presumed to capture non-specific signals. Subsequent quantitative analyses revealed that SWITCH-seq manifests an exemplary specificity, exceeding 99.5% (FIG. 5).

### Methods

Cell culture. The Human HeLa cell line was sourced from ATCC (CCL-2), and subsequent culturing was performed in DMEM supplemented with 10% FBS at 37° C. and 5% CO2.

SWITCH-seq experimental procedure. Glass-bottom 12-well plates (Mattek, P12G-1.5-14-F) were treated as follows: Oxygen plasma treatment was applied for 5 mins (Anatech Barrel Plasma System, 100W, 40% O₂), followed by sequential incubation with 1% methacryloxypropyltrimethoxysilane (Bind-Silane, GE Healthcare 17-1330-01) 88% ethanol (VWR, 89125-170), 10% acetic acid (Sigma-Aldrich, A6283-100ML), and 1% H₂O (Thermo Fisher Scientific, 10977023) at room temperature for 1 hour and 0.1 mg/mL Poly-D-lysine (Sigma-Aldrich, P7280-5X5MG) solution at room temperature for an additional hour. Micro cover glasses (Electron Microscope Sciences, 72226-01) underwent a pretreatment step with Gel Slick (Lonza, 50640) at room temperature for 15 mins and were then air-dried.

HeLa cells were cultured in treated 12-well plates, and after rinsing with 1×PBS (Thermo Fisher Scientific, 10010049), they were fixed with 1 mL of 1.6% PFA (Electron Microscope Sciences, 15710-S) in PBS buffer at room temperature for 15 mins. Following fixation, the cells underwent permeabilization by treatment with 1 mL of pre-chilled (−20° C.) methanol (Sigma-Aldrich, 34860-1L-R) and incubation at −20° C. for an hour. Thereafter, HeLa cells were transferred from the −20° C. fridge to room temperature for 5 mins, and then washed twice with PBSTR (0.1% Tween-20 (Calbiochem, 655206), 0.1 U/μL RNaseOUT™ (Thermo Fisher Scientific, 10777019) in PBS) for 5 mins each.

For the reverse transcription (RT) process, primers were prepared by dissolving them at a concentration of 250 μM in ultrapure RNase-free water (Thermo Fisher Scientific, 10977023), followed by pooling. All probes were manufactured by Integrated DNA Technologies (IDT). The probe mixture was subjected to heating at 90° C. for 5 mins, followed by cooling to room temperature. The samples were then treated with 300 μL of template switching mixture, which included 1× template switching buffer (New England Biolabs, M0466L), 250 μM dNTP (Invitrogen 100004893), 40 μM 5-(3-aminoallyl)-dUTP (Invitrogen AM8439), 2.5 μM RT primer, 0.4 U/μL RNaseOUT™, 3.3 μM template switching oligo, and 1× template switching RT enzyme mix. This mixture was incubated at 4° C. for 15 mins, followed by an overnight placement in a 42° C. humidified oven with gentle shaking.

The following day, the samples underwent three washes with 500 μL PBST (0.1% Tween-20 in PBS) for 5 mins each. To cross-link cDNA molecules containing aminoallyl-dUTP, the specimens were incubated with 5 mM BS (PEG)₉ (Thermo Fisher Scientific, 21582) in PBST for 1 hour at room temperature, followed by a wash with PBST at room temperature for 5 mins. The cross-linking reaction was quenched by treating the samples with 0.1 M Glycine (Sigma-Aldrich, 50046-250G) in PBST at room temperature for 30 mins. To 4911-6197-4934.1 48 degrade residual RNA and generate single-stranded cDNA, the specimens were incubated for 2 hours at 37° C. with an RNA digestion mixture, which was composed of 0.25 U/μL RNase H (New England Biolabs, M0297L), 1 mg/mL RNase A (Thermo Fisher Scientific, EN0531), and 10 U/μL RNase T1 (Thermo Fisher Scientific, EN0541) in 1×RNaseH buffer. The samples were then washed twice with PBST for 5 mins each. After the final PBST wash, the samples were incubated with 300 μL of splint ligation mixture containing 0.2 mg/mL BSA (New England Biolabs, B9000S), 2.5 μM splint ligation primer, and 0.1 U/μL T4 DNA ligase (Thermo Fisher Scientific, EL0011) in 1× T4 DNA ligase buffer at room temperature for 4 hours with gentle shaking. Subsequently, they were washed three times with 500 μL PBST for 5 mins each.

To create nanoballs of cDNA (amplicons) containing multiple copies of the original cDNA sequence, each cDNA circle undergoes linear amplification through rolling-circle amplification (RCA). This is achieved by immersing the cDNA in a 300 μL RCA mixture consisting of 0.2 U/μL Phi29 DNA polymerase (Thermo Fisher Scientific, EP0094), 250 μM dNTP, 40 μM 5-(3-aminoallyl)-dUTP, and 0.2 mg/mL BSA in 1×Phi29 buffer at 30° C. for 4 hours with gentle shaking. Following RCA, the samples were subjected to two washes with PBST. Subsequently, they were incubated with 20 mM methacrylic acid N-hydroxysuccinimide ester (Sigma-Aldrich, 730300-1G) in 100 mM sodium bicarbonate buffer at room temperature for one hour, followed by two additional washes with PBST for 5 mins each. The samples then experience a 10-minute incubation in 500 μL monomer buffer containing 4% acrylamide (Bio-Rad, 161-0140) and 0.2% bis-acrylamide (Bio-Rad, 161-0142) in 2×SSC (Sigma-Aldrich, S6639) at 4° C. Following the aspiration of the buffer, a 35 μL polymerization mixture, made of 0.2% ammonium persulfate (Sigma-Aldrich, A3678) and 0.2% tetramethylethylenediamine (Sigma-Aldrich, T9281) dissolved in monomer buffer, is placed at the core of the sample and is promptly covered with a Gel Slick-coated coverslip. The polymerization is then carried out inside an N₂ enclosure for 90 mins at room temperature. Afterward, the sample is washed three times with PBST, each time for 5 mins.

Several iterative sequencing experiments were conducted to decode the rRNA identity. For each iteration, the sample initially underwent treatment with a stripping buffer containing 60% formamide (Calbiochem, 655206) and 0.1% Triton-X-100 (Sigma-Aldrich, 93443) at room temperature twice for 10 mins each, followed by a triple wash in PBST, each lasting 5 mins. Then the samples were incubated with a 300 μL sequencing mixture containing 0.2 U/μL T4 DNA ligase, 0.2 mg/ml BSA, 10 μM reading probe, and 5 μM fluorescent decoding oligos in 1×T4 DNA ligase buffer for at least 3 hours at room temperature. Post-incubation, the samples were thrice washed with a washing and imaging buffer made of 10% formamide in 2×SSC buffer, each wash lasting for 10 mins. Following the washing steps, the samples were immersed in the washing and imaging buffer for imaging. DAPI (Sigma-Aldrich, D9542) was dissolved in wash and imaging buffer and performed following the manufacturer's instructions for nuclei staining for 20 mins. Images were captured using a Leica TCS SP8 confocal microscope equipped with a 40× oil immersion objective (NA 1.3) and an acquisition voxel size of 142 nm×142 nm×500 nm.

SWITCH-seq with hairpin TSO experimental procedure. After RNase digestion, the sample was incubated with 300 μL of gap-filling mixture containing 250 μM dNTP, 0.2 mg/ml BSA, and 0.2 U/μL Phusion® High-Fidelity DNA Polymerase (New England Biolabs, M0530L) in 1× Phusion® HF Buffer at 30 min at 37° C. followed by 45 min at 45° C. with gentle shaking. The samples were then washed twice with PBST for 5 mins each. After the final PBST wash, the samples were incubated with 300 μL of splint ligation mixture containing 0.2 mg/mL BSA and 0.1 U/μL T4 DNA ligase in 1×T4 DNA ligase buffer at room temperature for 3 hours with gentle shaking. Subsequently, they were washed three times with 500 μL PBST for 5 mins each. Then, the sample proceeds with cDNA generation as described above. Other procedures are the same as described above.

### Example 2: Applications of In Situ Ribosome Profiling

mRNAs achieve their biological function through translation into proteins. However, pure mRNA levels do not always correlate with translation levels17. Existing translatome mapping methods are either performed without spatial information18 or implemented in a gene-targeted fashion19. Here, it is shown that the SWITCH-seq method can be modified to achieve untargeted in situ ribosome profiling to map the spatial translatome at single-cell subcellular level. This is achieved by controlling the cDNA circularization step where only the cDNAs in proximity to a ribosome will be splint-ligated and circularized (FIG. 6A).

Briefly, before the RT step, a biotinylated rRNA-targeting splint probe is hybridized to the 18s rRNA. The rRNA-splint is associated with a ribonucleotide-containing blocker strand (rBlocker) to prevent its annealing to the RT primer. During the RT step, a random RT primer with a 5′ phosphorylated adapter serves as the RT primer, and a ribonucleotide-containing TSO (rTSO) with a BstZ17I recognition site is added. Then, streptavidin incubation and BS(PEG)₉ crosslinking are carried out to fix the rRNA-splint probe in place. Next, RNase digestion and BstZ17I restriction digestion are carried out to remove mRNA and rBlocker and generate a defined 3′ end of the rTSO. Then, splint ligation is performed to circularize all cDNAs in proximity to a ribosome. RCA primer hybridization followed by RCA is performed to amplify ribosome-bound cDNAs into amplicons. These amplicons may then be in situ sequenced via SEDAL20 or Illumina chemistry21.

This modified strategy was found to enrich ribosome-bound RNAs compared to the original SWITCH-seq method (FIGS. 6E-6F). Multiple variations of in situ ribosome profiling were also developed, including variations that employ a fixer strand to mitigate cDNA migration issues (FIG. 6B), utilize the ARTR-seq method22 to prepare cDNA from ribosome-bound mRNA (FIG. 6C), or directly recruit RT primer to ribosome-bound mRNA (FIG. 6D).

### Oligonucleotide Sequences

In nucleic acid sequences throughout the present disclosure, a nucleotide with an “r” directly preceding it indicates that this nucleotide is a ribonucleotide. A nucleotide without an “r” directly preceding it may be a ribonucleotide or a deoxyribonucleotide.

The notation “3InvdT” refers to the inclusion of a 3′ inverted deoxythymidine in the sequence.

The inclusion of the notation “+” in nucleotide sequences throughout the present disclosure indicates that the following nucleotide is a locked nucleic acid (LNA). An LNA is a modified RNA nucleotide in which the ribose moiety is modified with an extra bridge connecting the 2′ oxygen and 4′ carbon.

The inclusion of the notation “5ACryd” in sequences throughout the present disclosure refers to a 5′ acrydite modification. In some embodiments, acrydite comprises the structure

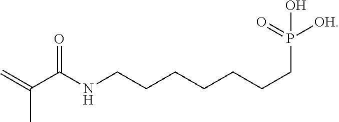 [SMILES: C=C(C)C(=O)NCCCCCCCP(=O)(O)O]

### Oligonucleotides Used in Example 1

| Oligo | |
|---|---|
| Name | Oligo Sequence |
| Reverse | |
| Transcription | |
| Primer | |
| RT_primer_ | /5Phos/TATACACTAAAGATAGGATCCACTAAAGGGGAGGAGGCGT |
| es151 | GGGGGG (SEQ ID NO: 9) |
| RT_primer_ | /5Phos/TATACACTAAAGATAGGATCCACTAAAAGGACGGGGAGCG |
| es271 | GGGGAGA (SEQ ID NO: 10) |
| RT_primer_ | /5Phos/TATACACTAAAGATAGGATCCACTAAAGGGGACCGGCCCG |
| es271_tandem | CGGCCCCT (SEQ ID NO: 11) |
| RT_primer_ | /5Phos/TATACACTAAAGATAGGATCCACTAAAGCTCTTCCCTGTTC |
| h11 | ACTCGCCGTTACT (SEQ ID NO: 12) |
| RT_primer_ | /5Phos/TATACACTAAAGATAGGATCCACTAAAGTGCGGTGCGTGAC |
| es391 | GGGC (SEQ ID NO: 13) |
| RT_primer_ | /5Phos/TATACACTAAAGATAGGATCCACTNNNNNNNN (SEQ ID NO: |
| linear | 14) |
| RT_primer_ | /5Phos/CACCGAGACTGCGAGTCACACATACACTAAAGCTTGGGAC |
| hairpin | AGACTACCTCTCTCGCAGTCTCGGTGATAGGATCCGCAGTNNNNN |
| | NNNCTCGCAGTCTCGGTG (SEQ ID NO: 15) |
| Reading | |
| Probe | |
| es271_R_1 | /5Phos/GGAGGGAGCGA/3InvdT/ (SEQ ID NO: 16) |
| es271_R_2 | /5Phos/GGGAGGGAGCG/3InvdT/ (SEQ ID NO: 17) |
| es271_tandem_ | /5Phos/CGAGGAGGAGG/3InvdT/ (SEQ ID NO: 18) |
| R_1 | |
| es271_tandem_ | /5Phos/CCGAGGAGGAG/3InvdT/ (SEQ ID NO: 19) |
| R_2 | |
| h11_R_1 | /5Phos/CTCCGCTGACT/3InvdT/ (SEQ ID NO: 20) |
| h11_R_2 | /5Phos/CCTCCGCTGAC/3InvdT/ (SEQ ID NO: 21) |
| es151_R_1 | /5Phos/CACCCCCGCCG/3InvdT/ (SEQ ID NO: 22) |
| es151_R_2 | /5Phos/ACACCCCCGCC/3InvdT/ (SEQ ID NO: 23) |
| es151_R_3 | /5Phos/GACCCCACACC/3InvdT/ (SEQ ID NO: 24) |
| es151_R_4 | /5Phos/GGACCCCACAC/3InvdT/ (SEQ ID NO: 25) |
| es391_R_1 | /5Phos/CGCGCCCCGTT/3InvdT/ (SEQ ID NO: 26) |
| es391_R_2 | /5Phos/CCGCGCCCCGT/3InvdT/ (SEQ ID NO: 27) |
| Linear_TSO_ | /5Phos/ATACACTAAAG/3InvdT/ (SEQ ID NO: 28) |
| reading_ | |
| seq | |
| Hairpin_TSO_ | /5Phos/ATCCCTGCCAAGCT/3InvdT/ (SEQ ID NO: 29) |
| reading_ | |
| seq | |
| TSO and | |
| split | |
| ligation | |
| probes | |
| Template | TACCAACGCAGAGTACATrGrG+G (SEQ ID NO: 30) |
| switching | |
| oligos | |
| (DNA) | |
| Template | rUrArCrCrArArCrGrCrArGrArGrUrArCrArUrGrG+G (SEQ ID NO: 31) |
| switching | |
| oligos | |
| (RNA) | |
| Template | rUrArCrCrArACGCrArGrArGrUrACATrGrG+G (SEQ ID NO: 32) |
| switching | |
| oligos | |
| (DNARNA | |
| hybrid) | |
| Template | AACCGAGACTGCGAGAAATACCCNNNNNCATCCCTGCCAAGCTTC |
| switching | TAACTCGCAGTCTCGGTTTCGTAGACTAAGATrGrG+G (SEQ ID NO: |
| oligos | 33) |
| (Hairpin) | |
| Splint | /5ACryd/AGTGGATCCTATCTTTAGTGTATATACCAACGCAGAGTAC |
| ligation | AT (SEQ ID NO: 34) |
| primer | |
| Fluorescent | |
| decoding | |
| oligos | |
| Decoding_ | /5Alex488N/NNNNNNAA |
| probe_1 | |
| Decoding_ | /5Alex488N/NNNNNNCC |
| probe_2 | |
| Decoding_ | /5Alex488N/NNNNNNGG |
| probe_3 | |
| Decoding_ | /5Alex488N/NNNNNNTT |
| probe_4 | |
| Decoding_ | /5Alex546N/NNNNNNAC |
| probe_5 | |
| Decoding_ | /5Alex546N/NNNNNNCA |
| probe_6 | |
| Decoding_ | /5Alex546N/NNNNNNGT |
| probe_7 | |
| Decoding_ | /5Alex546N/NNNNNNTG |
| probe_8 | |
| Decoding_ | /5Alex594N/NNNNNNAG |
| probe_9 | |
| Decoding_ | /5Alex594N/NNNNNNGA |
| probe_10 | |
| Decoding_ | /5Alex594N/NNNNNNCT |
| probe_11 | |
| Decoding_ | /5Alex594N/NNNNNNTC |
| probe_12 | |
| Decoding_ | /5Alex647N/NNNNNNAT |
| probe_13 | |
| Decoding_ | /5Alex647N/NNNNNNTA |
| probe_14 | |
| Decoding_ | /5Alex647N/NNNNNNGC |
| probe_15 | |
| Decoding_ | /5Alex647N/NNNNNNCG |
| probe_16 | |
| Other | |
| probes | |
| RNA | rArG rArGrC rCrArC rCrArU rGrGrU rGrArG rCrGrG rCrUrG rGrCrG |
| template | rGrCrU rGrUrU rCrArA rGrArA rGrArU rUrArG rCrUrG rArArA rArArA |
| for in vitro | rArArA rArArA rArArA rArArA rArArA (SEQ ID NO: 35) |
| template | |
| switching | |
| efficiency | |
| analysis | |

### REFERENCES

1. Ke, R. et al., In situ sequencing for RNA analysis in preserved tissue and cells. Nat. Methods 10, 857-860 (2013).
2. Wang, X. et al., Three-dimensional intact-tissue sequencing of single-cell transcriptional states. Science 361 (2018).
3. Chen, X., Sun, Y.-C., Church, G. M., Lee, J. H. & Zador, A. M. Efficient in situ barcode sequencing using padlock probe-based BaristaSeq. Nucleic Acids Res. 46, e22 (2018).
4. Femino, A. M., Fay, F. S., Fogarty, K., & Singer, R. H. Visualization of single RNA transcripts in situ. Science 280, 585-590 (1998).
5. Raj, A., van den Bogaard, P., Rifkin, S. A., van Oudenaarden, A., & Tyagi, S. Imaging individual mRNA molecules using multiple singly labeled probes. Nat. Methods 5, 877-879 (2008).
6. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
7. Lee, J. H. et al., Fluorescent in situ sequencing (FISSEQ) of RNA for gene expression profiling in intact cells and tissues. Nat. Protoc. 10, 442-458 (2015).
8. Qian, X. et al., Probabilistic cell typing enables fine mapping of closely related cell types in situ. Nat. Methods 17, 101-106 (2020).
9. Stickels, R. R. et al., Highly sensitive spatial transcriptomics at near-cellular resolution with Slide-seqV2. Nat. Biotechnol. 39, 313-319 (2021).
10. Rodriques, S. G. et al., Slide-seq: A scalable technology for measuring genome-wide expression at high spatial resolution. Science 363, 1463-1467 (2019).
11. Ståhl, P. L. et al., Visualization and analysis of gene expression in tissue sections by spatial transcriptomics. Science 353, 78-82 (2016).
12. Vickovic, S. et al., High-definition spatial transcriptomics for in situ tissue profiling. Nat. Methods 16, 987-990 (2019).
13. Zhu, Y. Y., Machleder, E. M., Chenchik, A., Li, R., & Siebert, P. D. Reverse transcriptase template switching: a SMART approach for full-length cDNA library construction. Biotechniques 30, 892-897 (2001).
14. Picelli, S. et al., Full-length RNA-seq from single cells using Smart-seq2. Nat. Protoc. 9, 171-181 (2014).
15. Rothschild, D. et al., A comprehensive rRNA variation atlas in health and disease. bioRxiv (2023) doi: 10.1101/2023.01.30.526360.
16. Wulf, M. G. et al., Chemical capping improves template switching and enhances sequencing of small RNAs. Nucleic Acids Res. 50, e2 (2022).
17. Vogel, C. & Marcotte, E. M. Insights into the regulation of protein abundance from proteomic and transcriptomic analyses. Nat Rev Genet 13, 227-232 (2012) https://doi.org: 10.1038/nrg3185.
18. VanInsberghe, M., van den Berg, J., Andersson-Rolf, A., Clevers, H., & van Oudenaarden, A. Single-cell Ribo-seq reveals cell cycle-dependent translational pausing. Nature 597, 561-565 (2021) https://doi.org: 10.1038/s41586-021-03887-4.
19. Zeng, H. et al., Spatially resolved single-cell translatomics at molecular resolution. Science 380, eadd3067 (2023) https://doi.org: 10.1126/science.add3067.
20. Wang, X. et al., Three-dimensional intact-tissue sequencing of single-cell transcriptional states. Science 361 (2018) https://doi.org: 10.1126/science.aat5691.
21. Metzker, M. L. Sequencing technologies—the next generation. Nat Rev Genet 11, 31-46 (2010) https://doi.org: 10.1038/nrg2626.
22. Xiao, Y. et al., Profiling of RNA-binding protein binding sites by in situ reverse transcription-based sequencing. Nat Methods 21, 247-258 (2024) https://doi.org: 10.1038/s41592-023-02146-w.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

1. A method for profiling RNA expression in a cell, the method comprising:
a) contacting the cell with a population of primer probes, wherein each primer probe in the population comprises a first oligonucleotide portion that is complementary to a portion of an RNA in the cell and a second oligonucleotide portion that is not complementary to the RNA;
b) contacting the cell with a reverse transcriptase, wherein the reverse transcriptase uses the first oligonucleotide portion of each of the primer probes to reverse transcribe the RNA to which each primer probe is hybridized, thereby producing a corresponding cDNA for each RNA;
c) contacting the cell with a template switching oligonucleotide (TSO), wherein a portion of the TSO is complementary to the 3′ end of each cDNA, and wherein the reverse transcriptase uses a portion of the TSO that is not complementary to the cDNA as a template to add the reverse complement sequence of the portion of the TSO to the 3′ end of the cDNA;
d) contacting the cell with an RNase, wherein the RNase digests all or substantially all of the RNA in the cell;
e) ligating the 5′ end and the 3′ end of each cDNA together to produce circular cDNA molecules;
f) performing rolling circle amplification to amplify the circular cDNA molecules, thereby producing a population of concatenated amplicons;
g) embedding the concatenated amplicons in a polymeric matrix; and
h) sequencing the concatenated amplicons, or a portion thereof, embedded in the polymeric matrix to determine the identity and location of RNAs in the cell.

2-4. (canceled)

5. The method of claim 1, wherein the first oligonucleotide portion of each of the primer probes comprises a random sequence.

6-8. (canceled)

9. The method of claim 1, wherein the first oligonucleotide portion of each of the primer probes comprises a known sequence.

10-24. (canceled)

25. The method of claim 1, wherein the step of ligating the 5′ end and the 3′ end of each cDNA together comprises contacting the cell with a circularization probe, wherein a portion of the circularization probe is complementary to the second oligonucleotide portion of the primer probe, and wherein another portion of the circularization probe is complementary to the reverse complement of the portion of the TSO added to the 3′ end of each cDNA.

26-29. (canceled)

30. The method of claim 1, further comprising contacting the cell with a splint probe comprising a binding moiety, an oligonucleotide portion that is complementary to an rRNA, an oligonucleotide portion that is complementary to the second oligonucleotide portion of a primer probe, and an oligonucleotide portion that is complementary to at least a portion of the sequence added to the 3′ end of the cDNA by the reverse transcriptase.

31-53. (canceled)

54. The method of claim 1, wherein the method is used to detect one or more mutations in an RNA, including a single base variant of an RNA.

55-61. (canceled)

62. The method of claim 1, wherein the polymeric matrix is a hydrogel.

63-67. (canceled)

68. The method of claim 1, wherein the sequencing is sequencing with error-reduction by dynamic annealing and ligation (SEDAL).

69. The method of claim 1, wherein RNA expression is profiled in multiple cells simultaneously.

70-72. (canceled)

73. The method of claim 1, wherein the cell is present within an intact tissue.

74-105. (canceled)

106. A set of oligonucleotides comprising:
i) a primer probe comprising a first oligonucleotide portion that is complementary to a portion of an RNA in a cell and a second oligonucleotide portion that is not complementary to the RNA; and
ii) a template switching oligonucleotide (TSO).

107. The set of oligonucleotides of claim 106 further comprising:
iii) a circularization probe, wherein a portion of the circularization probe is complementary to the second oligonucleotide portion of the primer probe, and wherein another portion of the circularization probe is complementary to the reverse complement of a portion of the TSO;
iv) a splint probe comprising a binding moiety, an oligonucleotide portion that is complementary to an rRNA, an oligonucleotide portion that is complementary to the second oligonucleotide portion of a primer probe, and an oligonucleotide portion that is complementary to at least a portion of the reverse complement of the TSO;
v) a blocking probe comprising a first RNA portion complementary to the portion of the splint probe that is complementary to the second oligonucleotide portion of a primer probe and a second RNA portion that is complementary to the portion of the splint probe that is complementary to the reverse complement of the TSO; and/or
vi) a fixer probe comprising a portion that is complementary to a portion of the primer probe and further comprising a binding moiety.

108. A plurality of oligonucleotides comprising multiple sets of oligonucleotides of claim 106, wherein each set of oligonucleotides comprises a primer probe that is complementary to a different RNA in a cell.

109. The plurality of oligonucleotides of claim 108, wherein the plurality comprises more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of oligonucleotides.

110. A kit comprising the set of oligonucleotides of claim 106.

111-119. (canceled)

120. A system for profiling RNA expression in a cell comprising:
a) a cell, tissue, or biological sample;
b) one or more primer probes, wherein each primer probe comprises a first oligonucleotide portion that is complementary to a portion of an RNA in the cell and a second oligonucleotide portion that is not complementary to the RNA;
c) a reverse transcriptase; and
d) a template switching oligonucleotide (TSO).

121. The system of claim 120 further comprising an RNase.

122. The system of claim 120, further comprising a DNA ligase.

123. The system of claim 120, further comprising a DNA polymerase.

124. (canceled)

125. The system of claim 120, further comprising reagents and monomers for preparing a polymeric matrix.

126-127. (canceled)


SEQUENCE LISTING (35 sequences)

SEQ ID NO: 1 - DNA, 24 bp, synthetic construct
  source 1..24: mol_type = other DNA
tatacactaaagataggatccact

SEQ ID NO: 2 - DNA, 65 bp, synthetic construct
  source 1..65: mol_type = other DNA
caccgagactgcgagtcacacatacactaaagcttgggacagactacctctctcgcagtctcggt

SEQ ID NO: 3 - protein, 560 aa, synthetic construct
AFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLGRLEDLKSKTLGDLVREAEKIFNKRETPEEREERIRRETEEKEERRRTVDEQKEKERDRRRHREMSKLLATVVIGQEQDRQEGERKRPQLDKDQCAYCKEKGHWAKDCPKKPRGPRGPRPQTSLLTLGDXGGQGQDPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVSLGFTWLSDFPQAWAESGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFR

SEQ ID NO: 4 - DNA, 21 bp, synthetic construct
  source 1..21: mol_type = other DNA
  misc_feature 1..18: note = DNA
  misc_feature 19..20: note = RNA
  misc_feature 21: note = DNA
taccaacgcagagtacatggg

SEQ ID NO: 5 - DNA, 21 bp, synthetic construct
  source 1..21: mol_type = other DNA
  misc_feature 1..20: note = RNA
  misc_feature 21: note = DNA
taccaacgcagagtacatggg

SEQ ID NO: 6 - DNA, 21 bp, synthetic construct
  source 1..21: mol_type = other DNA
  misc_feature 1..6: note = RNA
  misc_feature 7..9: note = DNA
  misc_feature 16..18: note = DNA
  misc_feature 19..20: note = RNA
  misc_feature 21: note = DNA
  misc_feature 10..15: note = RNA
taccaacgcagagtacatggg

SEQ ID NO: 7 - DNA, 80 bp, synthetic construct
  source 1..80: mol_type = other DNA
  misc_feature 1..77: note = DNA
  misc_feature 78..79: note = RNA
  misc_feature 80: note = DNA
aaccgagactgcgagaaatacccnnnnncatccctgccaagcttctaactcgcagtctcggtttcgtagactaagatggg

SEQ ID NO: 8 - DNA, 42 bp, synthetic construct
  source 1..42: mol_type = other DNA
agtggatcctatctttagtgtatataccaacgcagagtacat

SEQ ID NO: 9 - DNA, 46 bp, synthetic construct
  source 1..46: mol_type = other DNA
tatacactaaagataggatccactaaaggggaggaggcgtgggggg

SEQ ID NO: 10 - DNA, 47 bp, synthetic construct
  source 1..47: mol_type = other DNA
tatacactaaagataggatccactaaaaggacggggagcgggggaga

SEQ ID NO: 11 - DNA, 48 bp, synthetic construct
  source 1..48: mol_type = other DNA
tatacactaaagataggatccactaaaggggaccggcccgcggcccct

SEQ ID NO: 12 - DNA, 54 bp, synthetic construct
  source 1..54: mol_type = other DNA
tatacactaaagataggatccactaaagctcttccctgttcactcgccgttact

SEQ ID NO: 13 - DNA, 45 bp, synthetic construct
  source 1..45: mol_type = other DNA
tatacactaaagataggatccactaaagtgcggtgcgtgacgggc

SEQ ID NO: 14 - DNA, 32 bp, synthetic construct
  source 1..32: mol_type = other DNA
tatacactaaagataggatccactnnnnnnnn

SEQ ID NO: 15 - DNA, 103 bp, synthetic construct
  source 1..103: mol_type = other DNA
caccgagactgcgagtcacacatacactaaagcttgggacagactacctctctcgcagtctcggtgataggatccgcagtnnnnnnnnctcgcagtctcggtg

SEQ ID NO: 16 - DNA, 11 bp, synthetic construct
  source 1..11: mol_type = other DNA
ggagggagcga

SEQ ID NO: 17 - DNA, 11 bp, synthetic construct
  source 1..11: mol_type = other DNA
gggagggagcg

SEQ ID NO: 18 - DNA, 11 bp, synthetic construct
  source 1..11: mol_type = other DNA
cgaggaggagg

SEQ ID NO: 19 - DNA, 11 bp, synthetic construct
  source 1..11: mol_type = other DNA
ccgaggaggag

SEQ ID NO: 20 - DNA, 11 bp, synthetic construct
  source 1..11: mol_type = other DNA
ctccgctgact

SEQ ID NO: 21 - DNA, 11 bp, synthetic construct
  source 1..11: mol_type = other DNA
cctccgctgac

SEQ ID NO: 22 - DNA, 11 bp, synthetic construct
  source 1..11: mol_type = other DNA
cacccccgccg

SEQ ID NO: 23 - DNA, 11 bp, synthetic construct
  source 1..11: mol_type = other DNA
acacccccgcc

SEQ ID NO: 24 - DNA, 11 bp, synthetic construct
  source 1..11: mol_type = other DNA
gaccccacacc

SEQ ID NO: 25 - DNA, 11 bp, synthetic construct
  source 1..11: mol_type = other DNA
ggaccccacac

SEQ ID NO: 26 - DNA, 10 bp, synthetic construct
  source 1..10: mol_type = other DNA
cgcgccccgt

SEQ ID NO: 27 - DNA, 11 bp, synthetic construct
  source 1..11: mol_type = other DNA
ccgcgccccgt

SEQ ID NO: 28 - DNA, 11 bp, synthetic construct
  source 1..11: mol_type = other DNA
atacactaaag

SEQ ID NO: 29 - DNA, 14 bp, synthetic construct
  source 1..14: mol_type = other DNA
atccctgccaagct

SEQ ID NO: 30 - DNA, 21 bp, synthetic construct
  source 1..21: mol_type = other DNA
  modified_base 21: mod_base = OTHER; note = locked nucleic acid
  misc_feature 1..18: note = DNA
  misc_feature 19..21: note = RNA
taccaacgcagagtacatggg

SEQ ID NO: 31 - RNA, 21 nt, synthetic construct
  source 1..21: mol_type = other RNA
  modified_base 21: mod_base = OTHER; note = locked nucleic acid
taccaacgcagagtacatggg

SEQ ID NO: 32 - DNA, 21 bp, synthetic construct
  source 1..21: mol_type = other DNA
  modified_base 21: mod_base = OTHER; note = locked nucleic acid
  misc_feature 1..6: note = RNA
  misc_feature 7..9: note = DNA
  misc_feature 10..15: note = RNA
  misc_feature 16..18: note = DNA
  misc_feature 19..21: note = RNA
taccaacgcagagtacatggg

SEQ ID NO: 33 - DNA, 80 bp, synthetic construct; identical to SEQ ID NO: 7
  source 1..80: mol_type = other DNA
  modified_base 80: mod_base = OTHER; note = locked nucleic acid
  misc_feature 1..77: note = DNA
  misc_feature 78..80: note = RNA

SEQ ID NO: 34 - DNA, 42 bp, synthetic construct; identical to SEQ ID NO: 8
  source 1..42: mol_type = other DNA

SEQ ID NO: 35 - RNA, 68 nt, synthetic construct
  source 1..68: mol_type = other RNA
agagccaccatggtgagcggctggcggctgttcaagaagattagctgaaaaaaaaaaaaaaaaaaaaa